(19) United States
(12) Patent Application Publication
(10) Pub. No.: US 2007/0260174 A1
(43) Pub. Date: Nov. 8, 2007

(54) Detecting a failure to maintain a regimen

(75) Inventors: Edward K.Y. Jung, Bellevue, WA (US); Eric C. Leuthardt, St. Louis, MO (US); Royce A. Levien, Lexington, MA (US); Robert W. Lord, Seattle, WA (US); Mark A. Malamud, Seattle, WA (US); John D. Rinaldo JR., Bellevue, WA (US); Lowell L. Wood JR., Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC

(21) Appl. No.: 11/429,643

(22) Filed: May 5, 2006

(51) Int. Cl. A61M 31/00; A61K 9/22

(52) U.S. Cl. 604/65; 604/890.1

(57) ABSTRACT

One aspect relates to a technique for. analyzing at least one condition of at least one fluid(s) and/or element(s) as contained within an individual. The technique can include detecting a failure to maintain a regimen for the individual based at least in part on the analyzing the at least one condition of the at least one fluid(s) and/or element(s) as contained within the individual.

### TECHNICAL FIELD

Certain aspects of this disclosure can relate to, but are not limited to, a mechanism or technique that can detect a failure to maintain a regiment for an individual.

### BRIEF DESCRIPTION OF THE FIGURES

FIG. 1 is a block diagram of one embodiment of a detect failure to maintain regimen mechanism;

FIG. 2 is a diagram of an embodiment of the detect failure to maintain regimen mechanism;

FIG. 3 is a diagram of another embodiment of the detect failure to maintain regimen mechanism;

FIG. 4 is a diagram of another embodiment of the detect failure to maintain regimen mechanism;

FIG. 5 is a diagram of another embodiment of the detect failure to maintain regimen mechanism;

FIG. 6 is a diagram of another embodiment of the detect failure to maintain regimen mechanism;

FIG. 7 is a diagram of another embodiment of the detect failure to maintain regimen mechanism; and

FIG. 8 (including FIGS. 8a and 8b) is a flowchart representing one embodiment of a detect failure to maintain a regimen technique.

### DETAILED DESCRIPTION

At least certain portions of the text of this disclosure (e.g., claims and/or detailed description and/or drawings as set forth herein) can support various applications. Although, for sake of convenience and understanding, the detailed description can include section headings that generally track the titles of the various different supported applications or follow the general concepts contained therein, and is not intended to limit the scope of the invention as set forth by each particular claim. It is to be understood that support for the various applications thereby can appear throughout the text and/or drawings, irrespective of the section headings.

### 1. Certain Embodiments of a Detect Failure to Maintain Regimen Mechanism

Certain aspects of this disclosure can relate to a variety of a detect failure to maintain regimen mechanism 100, as well as a variety of techniques and/or operations associated therewith. Certain embodiments of the detect failure to maintain regimen mechanism 100 are configured to detect a failure of an individual to maintain a regimen. Certain embodiments of the detecting the failure to maintain regimen within the individual may be based, at least in part on a concentration or a relative percentage (e.g., by weight, volume, mass, etc. as may be appropriate for the particular treatment and/or regimen) of an at least one element(s) with respect to an at least one fluid(s), within the individual. Such failure to maintain a regimen may utilize a variety of aspects such as a result of failing to take appropriate treatment, continuing to provide treatment even when no longer necessary, failure to maintain a suitable level of the at least one element(s) in the at least one fluid(s) such as during illness, anesthesia, or injury, etc. Certain embodiments of the maintaining the regimen can thereby be associated with maintaining a desired level of an element within a fluid, in which the fluid is associated with the individual in such a manner as being contained within the individual.

Certain embodiments of the detect failure to maintain regimen mechanism 100 can be configured to operate on a near real-time basis, on a considerably slower basis, or as an as-desired basis, and can provide information relating to the individual to be monitored and/or reviewed on a basis limited only by the configuration, operation, and capabilities of the mechanism. Certain embodiments of the detect failure to maintain regimen mechanism 100 can be situated within the individual, and can monitor the condition of the at least one fluid(s) and/or element(s) of the individual, and can be configured or programmed to respond or treat the individual. Certain embodiments of at least a portion of the detect failure to maintain regimen mechanism 100 can be situated within the individual, and based at least in part on the condition of the at least one fluid(s) and/or element(s) can generate some monitoring information and/or data. The at least the portion of the detect failure to maintain regimen mechanism 100 can thereupon transmit the monitoring information and/or data to another portion of the detect failure to maintain regimen mechanism 100 and/or another device that can be configured to provide treatment. For example, certain embodiments of the monitoring information and/or data could be transmitted to an external monitoring and/or treating mechanism, similar to but not limited to the embodiments of devices as described in the following U.S. patent applications: U.S. patent application entitled MONITORING AND/OR TREATING SYRINGE MECHANISM, filed Apr. 27, 2006 to Jung et al.—Serial Number not Available (incorporated herein by reference in it's entirety); and U.S. patent application entitled REMOTE CONTROLLED IN VIVO REACTION METHOD, filed Nov. 9, 2005 to Hood et al., Ser. No. 11/272,572 (incorporated herein by reference in it's entirety). Certain embodiments of the detect failure to maintain regimen mechanism 100, as described in this disclosure, can include but are not limited to one or more of the following: an implant, a syringe feeding needle, a tube, a low-invasive device (such as a gas-liquid, or capsule injection device), etc.

Within this disclosure, the term “individual” such as can be treated at least partially using the detect failure to maintain regimen mechanism 100 can include and/or apply to a human, an animal, a plant, or an organism. As with numerous medical technologies, certain designs, uses, mechanisms, and/or configurations of the detect failure to maintain regimen mechanism 100 that is intended or designed to be applied to humans can, for example, also be applied intact, or with appropriate modifications, to animals or organisms (or vice versa).

Certain embodiments of the detect failure to maintain regimen mechanism 100 can be configured to analyze and/or detect at least one detected fluid(s) and/or element(s) that are contained with or associated with an individual 120. Certain embodiments of the detect failure to maintain regimen mechanism 100 can be configured to treat the individual 120 (such as maintaining the regimen in the individual) such as by monitoring the at least one fluid(s) and/or element(s) within the individual.

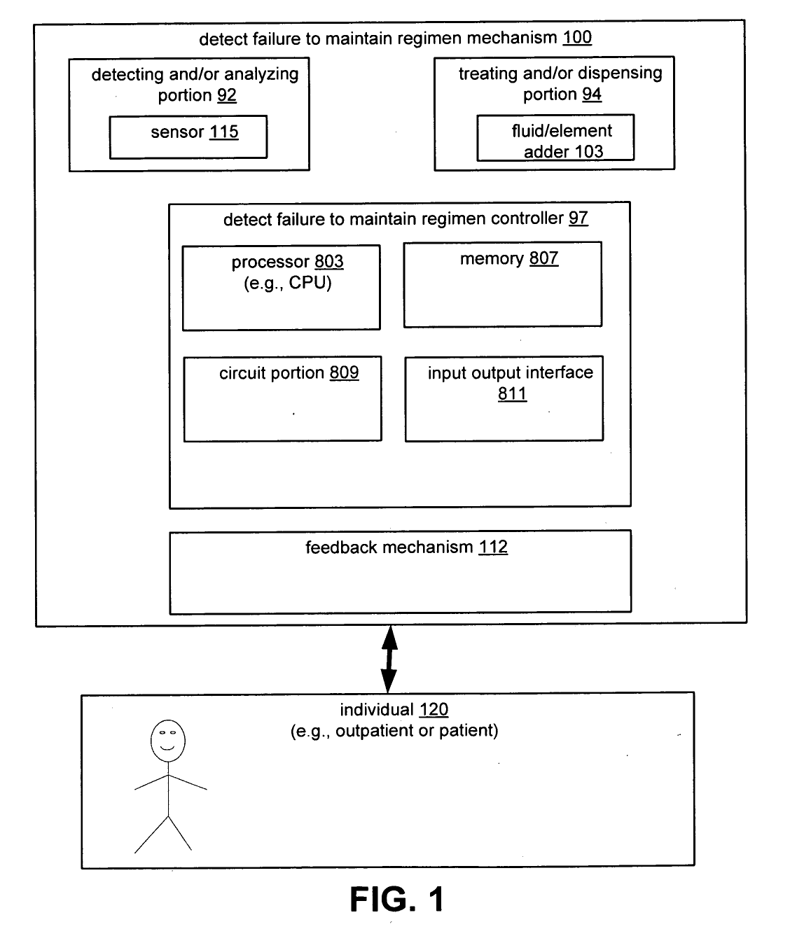

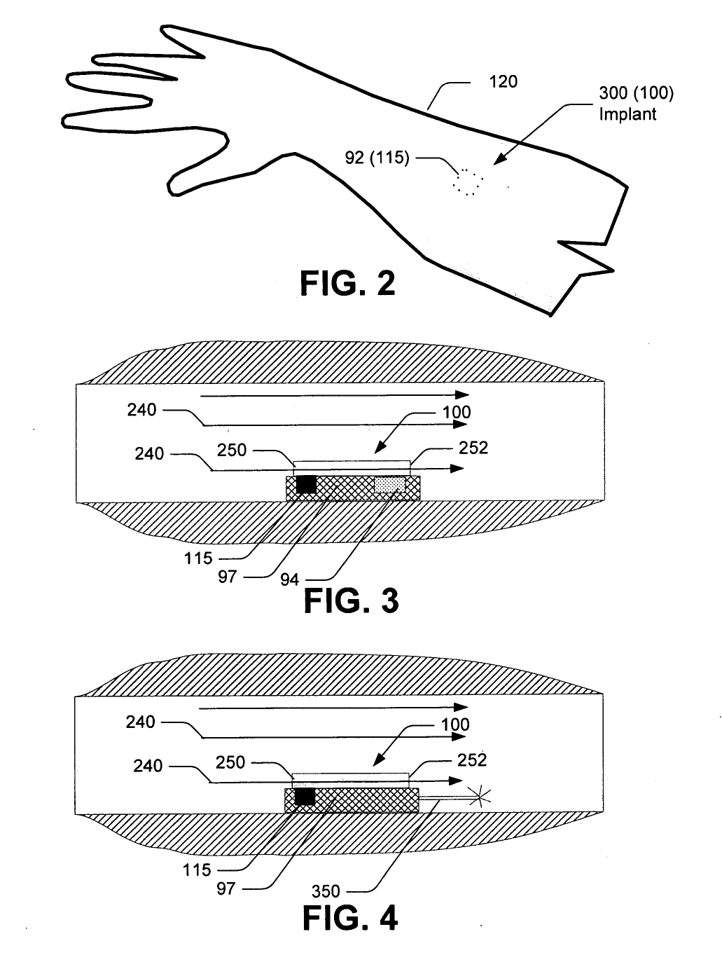

FIG. 1 shows a block diagram of one embodiment of the detect failure to maintain regimen mechanism 100 that can be used to perform one or more varied operations with respect to at least one fluid(s) and/or element(s) that can include, but is not limited to one or more of: detecting and/or analyzing the at least one fluid(s) and/or element(s) from the individual 120 and/or another source and/or treating the individual and/or dispensing the at least one fluid(s) and/or element(s) at least partially in response to the analyzing. There can be a variety of embodiments of the detect failure to maintain regimen mechanism 100 as described with respect to FIGS. 2 to 7, which may each be provided with distinct distinguishing reference characters in addition to 100.

Certain embodiments of the treating of the individual at least partially using the detect failure to maintain regimen mechanism 100 can include, but is not limited to, applying the at least one fluid(s) and/or element(s) into the individual 120 and/or establishing or providing a regimen (medical or other) for the individual. Certain embodiments of the detect failure to maintain regimen mechanism 100 can be a unitary mechanism, while other embodiments can be segmented into two or more devices. Each device can be configured to perform distinct, complementary, parallel, related, or unrelated operations. Certain embodiments of the detect failure to maintain regimen mechanism 100 can include a feedback mechanism 112, whereby the detecting the fluid(s) and/or element(s) that can be performed by certain embodiments of the treating and/or dispensing portion 94 can be at least partially performed by detecting, monitoring, and/or analyzing using certain embodiments of the detecting and/or analyzing portion 92. In certain embodiments, the operation of the detect failure to maintain regimen mechanism 100 can be altered and/or controlled at least partially in respect to the treating and/or dispensing portion 94. FIGS. 2 to 7 illustrate a number of the embodiments of the detect failure to maintain regimen mechanism 100 as described with respect to FIG. 1. Certain embodiments of the detect failure to maintain regimen mechanism 100 as described herein can be at least partially operationally, structurally, or functionally combined or run in parallel to treat or maintain the regimen for the individual.

Within this disclosure, the term “fluid” may, depending upon context, refer to liquids, gases, and other compositions, mixtures, or materials exhibiting fluid behavior. Within this disclosure, the term “fluid” can include, but is not limited to, the fluid that can at least partially be maintained within, analyzed relative to, obtained from, and/or applied to the individual 120. Certain embodiments of each of the at least one fluid(s) and/or element(s) (and/or a mixture thereof), can thereby include but is not limited to: one or more liquid(s), one or more solid(s), one or more gases, and/or one or more combination(s) thereof. Certain embodiments of the at least one fluid(s) and/or element(s) can be utilized for a variety of purposes relative to the individual that can include but are not limited to: associated with the metabolism of the individual, contained within the individual or an organ of the individual, maintaining the life of the individual, being a waste-product of the individual, or being some combination thereof.

Certain embodiments of the fluid within the body of the individual may thereby include a liquid, a gas, a mixture including a liquid, or a mixture or combination including a gas or a liquid. As used herein, the term fluid may encompass liquids, gases, or mixtures thereof that also include solid particles in a fluid carrier. Certain embodiments of the liquids may include, but are not limited to, mixtures of two or more different liquids, solutions, slurries, or suspensions. Examples of liquids present within the body can include blood, lymph, serum, urine, semen, digestive fluids, tears, amniotic fluid, ambiotic fluid, saliva, mucous, cerebro-spinal fluid, intestinal contents, bile, epithelial exudate, or esophageal contents. Certain embodiments of the liquids that can also be present within the body may include synthetic or introduced liquids, such as blood substitutes or drug, nutrient, or buffered saline solutions. Certain embodiments of the fluids may include liquids containing dissolved gases or gas bubbles, or gases containing fine liquid droplets or solid particles. Certain embodiments of the gases or gaseous mixtures found within the body may include inhaled and exhaled air, e.g. in the nasal or respiratory tract, or intestinal gases.

Within this disclosure, certain embodiments of the term “fluid” can include but is not limited to a “body fluid”. Body fluid components may include, but are not limited to, cells, cellular fractions or components, collections or aggregations of cells, bacterial, viral or fungal species, ions, molecules, gas bubbles, dissolved gas, suspended particles, or a variety of other materials that may be present in the body fluid, are also indicated. Body fluid components may be materials that are normally present in the body fluid, materials that are naturally derived but not normally present in the body fluid, or foreign materials that have entered or been introduced to the body fluid (including but not limited to pathogens, toxins, pollutants, or medications, for example).

Within this disclosure, the term “element” can include, but is not limited to, that which can be mixed with, combined with, integrated in, or otherwise associated with the fluid. For example, gasses, solids, liquids, chemicals, materials, and/or medicines, etc. that can be mixed, combined, suspended, or otherwise associated with the fluid may be considered individually or in combination as an embodiment of the element. Additionally, certain naturally-occurring chemicals, liquids, or gasses (e.g., carbon dioxide, oxygen, etc.), elements, viruses, germs, etc. at least partially contained within the fluid may be considered as an individual embodiment of the element. As such, certain embodiments of the at least one element can include a gas, a solid, or a liquid that can be included in, or carried by, the at least one fluid(s) and/or element(s). As such, within this disclosure, the terms “fluid” and “element” should be construed broadly, such as to indicate any fluid and/or element as associated with the individual which can be analyzed, detected, monitored, and/or treated.

Within this disclosure, the term “at least one fluid(s) and/or the at least one element(s)” can include, but is not limited to, one or more fluids, one or more elements, or any combination thereof. Within this disclosure, the term “at least one sensed fluid(s) and/or the at least one sensed element(s)” can include, but is not limited to, one or more fluids, one or more elements, or any combination thereof such as can be sensed by at least a portion of the detect failure to maintain regimen mechanism 100. Within this disclosure, the term “at least one modified fluid(s) and/or element(s)” can include, but is not limited to, one or more fluids, one or more elements, or any combination thereof that of the modified at least in part based upon any sensed condition of the individual. The term “at least one fluid(s) and/or the at least one element(s)” include, by definition, the at least one modified fluid(s) and/or element(s), the at least one monitored fluid(s) and/or element(s), and/or the at least one sensed fluid(s) and/or element(s), etc.

Within this disclosure, the term “at least one fluid(s) and/or element(s)”, or related terms, is not intended to follow typical antecedent basis rules. For instance, fluids and/or elements can be withdrawn from, injected into, combined with, modified, used to form, substituted by, used to establish a regimen or treatment to establish, the same or other element(s) and/or fluid(s) which can be similarly referenced as the “at least one fluid(s) and/or element(s)”. As such, the “at least one fluid(s) and/or element(s)” may not be limited to an identical fluid(s) and/or element(s).

Certain embodiments of the detecting the failure to maintain regimen can include, but are not limited to, ensuring that the amounts, concentrations, ratios, of the at least one fluid(s) and/or element(s) remains within a desirable or prescribed limit. Certain embodiments of the detecting failure to maintain the regimen can include detecting or monitoring the at least one fluid(s) and/or the at least one element(s) within the individual, such that certain aspects of the individual's condition, health, medication, sickness, etc. can be determined. As such, it is to be understood that the terms “fluid” is to be interpreted extremely broadly, and can be considered as applying even to fluids that could be included within an individual, and/or be applied via an implant.

Certain embodiments of the detect failure to maintain regimen mechanism 100 can be utilized or operated by the individual and/or a phlebotomist. Within this disclosure, the term “phlebotomist” or “phlebotomy” may be intended, depending upon context, to be applied to an individual (e.g., a patient or person utilizing the detecting or monitoring the treating mechanism) for whom the at least one fluid(s) and/or element(s) is maintained, monitored, treated, withdrawn and/or injected, etc. at least partially utilizing at least one implant, at least one needle, at least one solid capsule implant device, at least one fluid implant device, at least one patch, and/or another suitable detect failure to maintain regimen mechanism 100. The term “phlebotomist” or “phlebotomy” can also apply to the person using at least one embodiment of the detect failure to maintain regimen mechanism 100 to maintain, monitor, treat, maintain a regimen, and/or withdraw the at least one fluid(s) and/or element(s) relative to the individual. As such, certain embodiments of a phlebotomist can include, but is not limited to: a patient, a doctor, a nurse, an ambulance attendee, an emergency health-care provider, the individual, a friend or associate of the individual, a family member of the individual, etc.

Certain embodiments of the detect failure to maintain regimen mechanism 100 can be implanted. Certain embodiments of the detecting and/or monitoring as performed by the detect failure to maintain regimen mechanism 100 can be performed either remotely or at the detect failure to maintain regimen mechanism 100.

Certain embodiments of the detect failure to maintain regimen mechanism 100, as described with respect to FIG. 1, can include, but is not limited to: a detecting and/or analyzing portion 92, a treating and/or dispensing portion 94, a detect failure to maintain regimen controller 97, and/or a feedback mechanism 112. Certain embodiments of the detecting and/or analyzing portion 92 can include a sensor 115 that can be configured to analyze or detect the condition of the at least one fluid(s) and/or element(s) that can be maintained within, analyzed within, withdrawn from, injected into, or otherwise applied to the individual. Within this disclosure, the terms “condition” relating to the individual can include, but is not limited to: an illness, a sickness, an organism, a cancer, an immune deficiency, etc., or other such conditions that can be detected, monitored, analyzed, and/or treated at least partially using certain embodiments of the detect failure to maintain regimen mechanism 100. Within this disclosure, the term “condition” relating to the at least one fluid and/or element can include but is not limited to an aspect, condition, concentration, relative concentration, or other factor of the at least one fluid(s) and/or element(s).

Certain embodiments of the sensor 115 can include, but is not limited to: an optical sensor, an imaging device, an acoustic sensor, a pressure sensor, a temperature sensor, a flow sensor, a viscosity sensor, a shear sensor, or another suitable sensor that can be configurable to detect a condition or parameter of the at least one fluid(s) and/or element(s). Certain instances of the detecting the condition of the at least one fluid(s) and/or element(s) can include, but is not limited to, such operations as measuring the effective shear modulus of the fluid at a frequency or strain-rate, a chemical sensor for determining the concentration of a chemical compound or species, a biosensor, and an electrical sensor, an optical sensor, or another sensor configurable to sense a desired parameter or characteristic, for example. Certain embodiments of an optical sensor may be configured to measure the optical absorption, optical emission, fluorescence, electromagnetic radiated commissions, or phosphorescence of at least a portion of the fluid, for example. Such optical properties may be inherent optical properties of all or a portion of the fluid, or may be optical properties of materials added or introduced to the fluid, such as tags or markers for materials of interest within the fluid. Certain embodiments of a biosensor may detect materials including, but not limited to, a biological marker, an antibody, an antigen, a peptide, a polypeptide, a protein, a complex, a nucleic acid, a cell (and, in some cases, a cell of a particular type, e.g. by methods used in flow cytometry), a cellular component, an organelle, a gamete, a pathogen, a lipid, a lipoprotein, an alcohol, an acid, an ion, an immunomodulator, a sterol, a carbohydrate, a polysaccharide, a glycoprotein, a metal, an electrolyte, a metabolite, an organic compound, an organophosphate, a drug, a therapeutic, a gas, a pollutant, or a tag. Certain embodiments of a biosensor may include an antibody or other binding molecule such as a receptor or ligand. Certain embodiments of the sensor 115 may include a single sensor, while other embodiments of the sensor can include an array of sensors. Certain embodiments of the sensor may not be limited to a particular number and/or type of sensor. Certain embodiments of the sensor 115 might comprise in part or whole, a gas sensor such as an acoustic wave sensor, a chemiresistant sensor, a piezoelectric sensor, a liquid sensor, an electromechanical sensor, or perhaps an electronic nose.

Certain embodiments of the sensor 115 may be very small, comprising a sensor or array that can be a chemical sensor (Chemical Detection with a Single-Walled Carbon Nanotube Capacitor E. S. Snow, 2005 Science Vol. 307; 1942-1945), a gas sensor (Smart single-chip gas sensor microsystem Hagleitner, C. et al. 2001 NATURE VOL 414p. 293-296), an electronic nose, a nuclear magnetic resonance imager (“Controlled multiple quantum coherences of nuclear spins in a nanometre-scale device”, Go Yusa, 2005, Nature 343: 1001-1005) or other type of sensor. Considering that certain embodiments of the sensor 115 may be implanted in the individual, those embodiments of sensor should be resistant to attack (or encased to protect against attack) by internal chemicals, liquids, elements, antibodies, etc. Further examples of sensors that can be utilized as the sensor 115 are provided in The Biomedical Engineering Handbook, Second Edition, Volume I, J. D. Bronzino, Ed., Copyright 2000, CRC Press LLC, pp. V-1-51-9, and U.S. Pat. No. 6,802,811, both of which are incorporated herein by reference. Certain embodiments of the sensor 115 may be configured to measure various parameters, including, but not limited to, the electrical resistivity of the fluid, the density or sound speed of the fluid, the pH, the osmolality, or the index of refraction of the fluid of at least one wavelength. The selection of a suitable sensor for a particular application or use site is considered to be within the capability of a person having skill in the art.

At least certain ones of the operation(s) that can be performed by the detecting and/or analyzing portion 92, as well as at least certain ones of the operation(s) that can be performed by the treating and/or dispensing portion 94, can be performed entirely by certain embodiments of the detect failure to maintain regimen mechanism 100. Alternatively, at least certain ones of the operation(s) that can be performed by the detecting and/or analyzing portion 92 as well as at least certain ones of the operation(s) that can be performed by the treating and/or dispensing portion 94 can be performed by multiple such modular units that can include but is not limited to certain embodiments of the treating and/or dispensing portion 94 of the detect failure to maintain regimen mechanism 100.

FIG. 1 shows one embodiment of the detect failure to maintain regimen mechanism 100 that can include, among other portions, the detecting and/or analyzing portion 92 and/or the treating and/or dispensing portion 94. Certain embodiments of the detecting and/or analyzing portion 92, and a variety of techniques associated therewith as described with respect to this disclosure, can be configured to detect and/or analyze the at least one fluid(s) and/or element(s) associated with and/or contained within the individual 120. In certain instances, the at least one individual whose at least one fluid(s) and/or at least one element(s) are being analyzed can be a human, or alternately can be an animal or organism.

The embodiment of the detecting and/or analyzing portion 92 that can be used as described with respect to FIG. 1 can perform a variety of functions with respect to the at least one fluid(s) and/or at least one element(s), which can include but is not limited to: obtaining the at least one fluid(s) and/or element(s) either within and/or outside of the individual and/or analyzing the at least one fluid(s) and/or element(s). Certain embodiments of the obtaining the at least one fluid(s) and/or element(s) can be based at least in part on the analysis, which can be performed by the detecting and/or analyzing portion 92, while other embodiments can be performed by the treating and/or dispensing portion 94. For example, certain embodiments of the detecting and/or analyzing portion 92 can be configured to maintain within the individual, withdraw, aspirate, and/or analyze the at least one fluid(s) and/or element(s), and based upon the analysis, modify the at least one fluid(s) and/or element(s) to derive the at least one modified fluid(s) and/or element(s), etc. Certain embodiments of the detecting and/or analyzing portion 92 can be configured to analyze the at least one fluid(s) and/or element(s) at least partially within the individual, and based upon the analysis, modify the at least one fluid(s) and/or element(s) to derive the at least one modified fluid(s) and/or element(s), etc. As such, a particular embodiment of the detect failure to maintain regimen mechanism 100 can be configured or utilized to perform each of these functions; only certain ones of these functions; each of these functions plus additional functions; or only certain ones of these functions plus additional functions. Certain embodiments of the detecting and/or analyzing portion 92 can be situated structurally or operationally entirely within the individual, partially within the individual, or entirely outside of the individual.

Certain embodiments of the treating and/or dispensing portion 94 can be configured to provide treatment for a detected illness, sickness, etc. such as by dispensing the at least one fluid(s) and/or element(s). Such detecting, monitoring, and/or analysis can be performed on a variety of the at least one fluid(s) and/or element(s) relating to each particular illness, sickness, etc. based, for example, on the treatment and/or regimen. Certain embodiments of the detect failure to maintain regimen mechanism 100, as described with respect to FIG. 1, can include a feedback mechanism 112 that can detect or monitor a condition of the individual and/or the at least one fluid and/or element of the individual, and provide a suitable treatment or regimen of treatment at least partially in response to the detected or monitored condition. Certain embodiments of the feedback mechanism can thereby be utilized at a location or region remote of a physician, hospital, clinic, etc.; and can thereby be considered to provide an adoptive medical treatment or regimen.

Certain embodiments of the treating and/or dispensing portion 94 can be configured to, at least partially based upon the analysis, inject or provide with the at least one fluid(s) and/or element(s) into the individual. At least certain portions or certain embodiments of the treating and/or dispensing portion 94 can be configured as an implant as described in this disclosure. Certain embodiments of the treating and/or dispensing portion 94 can be situated structurally or operationally entirely within the individual, partially within the individual, or entirely outside of the individual.

A variety of embodiments of the detect failure to maintain regimen mechanism 100 can be configured or utilized to perform a variety of the following operations, only certain ones of the operations, each of these operations plus additional functions, or only certain ones of these operations and additional functions. Certain embodiments of the detect failure to maintain regimen mechanism 100 can at least be partially configured to analyze the at least one fluid(s) and/or element(s) at least partially within the individual. Another portion of the detect failure to maintain regimen mechanism 100 (or another detect failure to maintain regimen mechanism 100) can, at least partially based upon the analysis, dispense or apply the at least one fluid(s) and/or element(s) into the individual. Certain other embodiments of the detect failure to maintain regimen mechanism 100 can perform only certain ones of these operations, and may interact with other embodiments of the detect failure to maintain regimen mechanism that can, for example, perform certain prescribed operations as described in this disclosure. Certain embodiments of the detect failure to maintain regimen mechanism 100 can be configured to analyze the at least one fluid(s) and/or element(s) within the individual, and at least partially based upon the analysis, provide or dispense at least one modified fluid(s) and/or element(s) within the individual. As such, certain embodiments of the detect failure to maintain regimen mechanism 100 can perform a variety of, or all of the operations, associated with the detect failure to maintain regimen mechanism 100.

Certain embodiments of the detect failure to maintain regimen mechanism 100 can perform only certain ones of the operations, and as such can interface with other embodiments and/or portions of the detecting, monitoring, and/or treating application. For example, certain embodiments of the detect failure to maintain regimen mechanism 100 such as is at least partially internal to the individual 120, as illustrated with respect to FIG. 4 can detect, monitor, and/or analyze the at least one fluid(s) and/or element(s) within the individual, and transmit information relating thereto to another embodiment of the detect failure to maintain regimen mechanism 100. Thereupon, another portion of the detect failure to maintain regimen mechanism 100 (or another detect failure to maintain regimen mechanism 100) as described with respect to FIGS. 2 to 7 may alternately treat the individual or maintain the regimen to the individual; or alternately may transfer the results of the detecting, monitoring, and/or analysis to another portion of the detect failure to maintain regimen mechanism 100 (or alternately to another detect failure to maintain regimen mechanism 100 that may be at least partially situated outside of the individual 120) that can treat the individual or maintain the regimen. As such, the results of the detecting, monitoring, and/or analysis can be alternately treated within the individual with certain embodiments of the detect failure to maintain regimen mechanism 100; or alternately transferred to a variety of different embodiments of the detect failure to maintain regimen mechanism 100 as described with respect to FIGS. 1, 2, 3, 4, 5, 6, and/or 7. Thereupon, certain embodiments of the detect failure to maintain regimen mechanism 100 can be configured to determine the at least one fluid(s) and/or element(s) that should be added to, applied to, or injected into the individual for treatment, and thereupon inject, apply and/or add the at least one modified fluid(s) and/or element(s) into the individual based at least in part on the detecting, monitoring, and/or analysis.

Certain embodiments of the structural or operational elements depicted in FIGS. 1-7 of the detect failure to maintain regimen mechanism 100 are intended to serve as examples, and are in no way limiting. Certain embodiments of the structural elements are configured to provide physical support for components of the detect failure to maintain regimen mechanism 100. The choice of the particular structural element size and configuration as to be appropriate for the particular body of the individual may be selected by a person of skill in the art. Certain embodiments of the structural elements may be constructed using a variety of manufacturing methods, from a variety of materials. Appropriate materials may include metals, ceramics, polymers, and composite materials. Certain embodiments of the material selected should have suitable biocompatibility, sterilizability, mechanical, and physical properties, as is known to those of skill in the art. Examples of materials and selection criteria are described, for example, in The Biomedical Engineering Handbook, Second Edition, Volume I, J. D. Bronzino, Ed., Copyright 2000, CRC Press LLC, pp. IV-1-43-31. Certain embodiments of the structural elements may both provide relative support for and enclose one or more of the operational components, while certain embodiments of the structural elements may only provide a support for but not enclose at least certain operational components. Manufacturing techniques may include injection molding, extrusion, die-cutting, rapid-prototyping, etc., and will depend on the choice of material and device size and configuration. Sensing and active portions of the lumenally-active device as well as associated electrical circuitry (not depicted in FIGS. 1-7) may be fabricated on the structural element using various microfabrication and/or MEMS techniques, or may be constructed separately and subsequently assembled to the structural element, as one or more distinct components. Such design aspects of the structural components would generally be understood by those skilled in the art, and will not be further detailed herein.

Certain embodiments of the detect failure to maintain regimen mechanism 100 can be configured to analyze the at least one fluid(s) and/or element(s) that can be maintained within the individual (e.g., without providing any of the at least one fluid(s) and/or element(s) within the individual). In certain embodiments, another detect failure to maintain regimen mechanism that is situated at least partially external to the individual can utilize the information, instructions, etc. from another portion or embodiment of the detect failure to maintain regimen mechanism, and thereupon add the at least one modified fluid(s) and/or element(s) into the individual based at least in part on the analysis. As such, certain embodiments of the detect failure to maintain regimen mechanism 100 can be segmented into “modular” portions or units, each of which are configurable or able to perform only certain of the aspects of the entire detect failure to maintain regimen mechanism.

Certain operations or tasks performed by certain embodiments of the detect failure to maintain regimen mechanism 100 may not be performed by other embodiments of the detect failure to maintain regimen mechanism, or alternately may be performed by a user of the detect failure to maintain regimen mechanism. For example, based upon input (analysis of the at least one fluid(s) and/or element(s) which may be provided using a variety of user interfaces or information displays) by certain embodiments of the detect failure to maintain regimen mechanism 100, a user such as a phlebotomist or the individual may decide to inject the at least one modified fluid(s) and/or element(s) into the individual.

In certain embodiments, the at least one fluid(s) and/or element(s) that can be analyzed relative to the individual may be the type of fluid(s) and/or element(s) that can maintain the life of the individual, such as blood, air, water, etc. When such operations on life-maintaining fluid(s) and/or element(s) as withdrawing, analyzing, modifying, or injecting into the individual should be performed in a careful fashion as to not harm the individual. For example, only certain limited volumes of blood or air should be removed from, applied to, or modified within the individual to limit the possibility of shock to the individual, etc. Additionally, care should be applied to providing consistent operations with such life-maintaining fluids as blood, such as by ensuring consistent blood types are utilized, ensuring that too much blood is not withdrawn, etc. Certain embodiments of the detect failure to maintain regimen mechanism 100 can thereby detect the treatment of certain types of individuals such as to ensure that the treatment is being properly provided.

Certain embodiments of the detect failure to maintain regimen mechanism 100 can be utilized to assist in or improve accepted phlebotomy techniques. For example, certain embodiments of the detect failure to maintain regimen mechanism 100 can be used to assist in checking, and in certain instances limiting, these instances where inconsistent or incorrect medical treatment may be attempted to be provided. For example, certain embodiments of the detect failure to maintain regimen mechanism as described in this disclosure could analyze the withdrawn or aspirated blood type, and based on knowledge of the blood to be transfused, ensure that inconsistent blood types and/or components are not combined with the withdrawn blood or components and/or injected back into the individual. A variety of allergies, reactions, illnesses, etc. of each individual could be similarly checked using the detect failure to maintain regimen mechanism. Also, if a particular detect failure to maintain regimen mechanism is being configured for a particular individual, the detect failure to maintain regimen mechanism could be configured to ensure it is being applied to that type of individual.

Certain embodiments of the detect failure to maintain regimen mechanism 100 may be configured, depending upon context, to assist the individual in maintaining a medical regimen such as might have been established by the physician or other medical personnel. Consider that after a physician may have prescribed a particular treatment for certain ones of their individuals and/or patients, then in many instances the individuals and/or patients may fail to follow up on their treatment and/or regimen. The reasons why the individuals and/or patients may fail to follow up on treatments or regimens may include, but is not limited to: complexity of treatment, expense of treatment, requirement to travel to remote locations for treatment (e.g., a doctor's office, clinic, hospital, etc.), the individual's time spent associated within the doctor's office, clinic, hospital, etc. in receiving the treatment, failure of the individual in understanding how to maintain the treatment or regimen, etc. As such, it may be difficult for the individual to remain on the medical treatment and/or regimen for a variety of reasons. It would be highly desirable to provide a feedback mechanism to automate the treatment or maintaining the regimen as much as practicable, and thereby limit the effects of human inattentiveness or forgetfulness on the individual's treatment and/or regimen. Certain embodiments of the detect failure to maintain regimen mechanism 100 can also monitor the effect the treatment might be having on the regimen, and adjust the treatment, medication, or regimen accordingly.

Certain embodiments of the detect failure to maintain regimen mechanism 100 can be configured to make it easier to maintain their medical regimen, treatment, etc. for a particular individual. One aspect of maintaining the medical regimen, treatment, etc. can be to detect and/or analyze the individual's (or the fluid(s) and/or element(s) of the individual) response to the medical regimen, treatment, etc. As such, certain embodiments of the detect failure to maintain regimen mechanism can be configured to detect a condition of the individual at least in part by analyzing the at least one fluid(s) and/or element(s) obtained from the individual as described in this disclosure; and/or analyzing elements of the and/or included in the at least one fluid(s) and/or element(s) as described in this disclosure. Certain embodiments of the detect failure to maintain regimen mechanism therefore allow an individual (and/or an associated phlebotomist) to detect the condition or concentration of the at least one fluid(s) and/or element(s) in a manner that in many instances involving traditional medical techniques could only be performed in a medical office, hospital, etc.

Certain embodiments of the detect failure to maintain regimen mechanism can include some type of a detecting communication interface, by which the detecting or analyzed condition of the individual, the at least one fluid(s) included within the individual, and/or one or more element(s) included within the at least one fluid(s) and/or element(s) included within the individual can be communicated to a remote location such as where physicians or other health-care individuals can be situated. Thereupon, in certain embodiments, a physician or other health-care individual can respond by, for example, analyzing the detected condition of the individual, their fluid(s), and/or their element(s); and thereupon providing information, data, and/or instructions that could be utilized by the detect failure to maintain regimen mechanism 100 using a variety of user-interface techniques, communication techniques, and/or networking techniques. For example, a remotely situated physician or other medical personnel could provide information or instructions indicating if the detect failure to maintain regimen mechanism 100 should operate by applying, or not applying, the at least one fluid(s) and/or element(s) to the individual. For example, in certain embodiments, the detect failure to maintain regimen mechanism 100 can inject the modified contents and the at least one fluid(s) and/or element(s) into the individual based at least in part in certain instances on remote instructions.

With certain embodiments of the detect failure to maintain regimen mechanism 100, the physician or other health-care individual can be remotely situated while providing and detecting a suitable treatment and/or regimen to the individual. Therefore, by using certain embodiments of the detect failure to maintain regimen mechanism 100, the individual does not have to take time out of their schedule to visit the doctor or a hospital or clinic, as might otherwise be used to ensure the treatment or the regimen is being followed and/or considering how the individual's condition is changing or responding in response to the regimen or treatment. Such treatment and/or regimen as provided by certain embodiments of the detect failure to maintain regimen mechanism 100 can consider or adapt to one or more conditions of the individual and/or a condition of the at least one fluid(s) and/or element(s). As such, the conditions of the individual and/or a condition of the at least one fluid(s) and/or element(s) can be taken into consideration when providing the treatment and/or regimen. A considerable amount of the health-care detecting and/or treatment can thereby be provided remotely of the physician or other health-care individual utilizing certain embodiments of the detect failure to maintain regimen mechanism 100.

Certain embodiments of the detect failure to maintain regimen mechanism 100 can be configured to remotely detect and/or treat individuals in a manner that may comply with the regimen. Certain embodiments of the detecting and/or treating can be provided by the detect failure to maintain regimen mechanism 100 such as to be provided remotely from a physician, medical personnel, that has created a treatment and/or regimen for the individual. Certain embodiments of the treatment or regimen such as provided by certain embodiments of the detect failure to maintain regimen mechanism 100 can be responsive to the condition of the individual, and/or the condition of their at least one fluid(s) and/or element(s).

There may be a variety of treatments, illnesses, injuries, etc. at which a considerable portion of the treatment and/or regimen involves detecting a condition or concentration of the individual, a condition of the at least one fluid(s), and/or detecting a condition of the at least one element(s) associated with or included in the at least one fluid(s) and/or element(s). For example, such treatments and/or regimens can be provided for but are not limited to such conditions, elements, or disease is as, e.g., diabetes, flu, colds, AIDS, cancer, heart treatment medicine, etc. In certain instances, it may be desirable to provide such treatments or regimens in remote locations from a clinic, hospital, doctor's office, etc. at which remote location the individual cannot be easily detected. Such treatments and/or regimens can be provided remotely using certain embodiments of the detecting or feedback techniques and/or mechanisms as described in this disclosure. In certain instances, travel by the individual to a traditional health-care provider may be sufficiently difficult, time-consuming, or expensive to limit the individual's participation in the treatment and/or regimen. Certain countries and/or regions exist at which the individuals (as a result perhaps of financial difficulty, difficulty in medical treatment access, and/or lack of medical education) may not pursue suitable medical treatment as and/or medical regimens. These aspects, as well as others, can explain why a considerable percentage of traditional treatments and/or regimens prescribed by doctors are not followed by the individuals or patients.

Certain embodiments of the detect failure to maintain regimen mechanism 100 may be configurable to provide limited or no fluid(s) and/or element(s) in those instances that it determines the individual doesn't need the regimen, such as if they are analyzed being “cured”, or at least within prescribed limits. For instance, if the detecting and/or analyzing portion 92 determines that the suitable treatment for the individual is not to modify the at least one fluid(s) and/or element(s), then certain embodiments of the treating and/or dispensing portion 94 of the detect failure to maintain regimen mechanism 100 may act appropriately (since treatment may not be necessary) by, e.g., by not medicating, providing, or otherwise modifying the at least one fluid(s) and/or element(s) of the individual. As such, certain embodiments of maintaining the regimen for the individual may include not modifying the at least one fluid(s) and/or element(s). Within the individual by not adding, removing, or modifying the least one fluid(s) and/or element(s).

Certain embodiments of the detect failure to maintain regimen mechanism 100 may be particularly adaptable to emergency or accident situations. Certain emergency or accident situations may exist, such as hurricanes, landslides, volcanoes, etc. where access to such medical treatments and/or regimens (or medical personnel who can apply such medical treatments and/or regimens) may be difficult or impossible. Certain embodiments of the detect failure to maintain regimen mechanism 100, for example, can be pre-configured to detect failure to maintain a regimen for (such as at least partially by analyzing the condition of) the individual and/or the at least one fluid(s) and/or element(s) that might be associated with a particular epidemic, for example. Certain embodiments of the detect failure to maintain regimen mechanism 100 can be ingested, injected, applied using a nasal spray, applied using a patch, applied using a surgical port, implanted, positioned at a suitable position using a scope-type device, etc. Each embodiment of the detect failure to maintain regimen mechanism 100 should be configured to survive based at least in part on its intended location of operation. Certain embodiments of the detect failure to maintain regimen mechanism 100 can allow for providing a suitable detected treatment and/or regimen that are situated at locations remote from medical personnel, or which would be difficult for medical personnel to reach (e.g., to particular illnesses, sicknesses, etc. such as may be prevalent in those conditions) such as in certain emergency or accident situations.

Certain embodiments of the detect failure to maintain regimen mechanism 100 can limit the necessity of the individual to travel to the health-care provider and/or physician. As such, certain embodiments of the detect failure to maintain regimen mechanism 100 can be configured to detect, analyze and/or treat conditions likely to be encountered by such victims of particular emergency or accident situations such as could be easily detected. The emergency situations could include, but are not to necessarily limited to: diseases, flu, epidemics, and/or other treatable diseases. The medical capabilities of the individuals applying these emergency-based embodiments of the detect failure to maintain regimen mechanism 100 can be considerably less than that of other implants and/or syringes, and as such aid workers could in certain instances provide rapid reliable aid to emergency or accident victims.

Certain embodiments of the detect failure to maintain regimen mechanism 100 may be configured to provide particular medical treatments, situations, or regimens. With certain embodiments of traditional treating regimens, for example, the individual being treated (e.g., a patient) can obtain a responsive treating regimen by visiting a hospital, clinic, doctor's office, etc. at which the condition of the individual, their at least one fluid(s) and/or element(s) can be detected. For instance, a physician or other medical personnel can prepare a treatment and/or regimen that can vary depending upon whether some condition of the individual remains within certain prescribed limits. In certain circumstances when the condition of the individual (and/or the condition of their at least one fluid(s) and/or element(s)) falls outside of the prescribed limits, then the treatment and/or regimen can be suitably altered using the feedback mechanism 112, as described with respect to FIG. 1. One challenge with certain embodiments of the traditional treatment and/or regimen is to detect the condition of the individual, their at least one fluid(s) and/or element(s) such as to provide a feedback mechanism for the treatment and/or regimen based at least in part on the condition of the individual and/or their at least one fluid(s) and/or element(s). Certain embodiments of the feedback mechanism can thereby detect how the individual is responding to a particular treatment or regimen, and/or the condition of the individual.

Additionally certain traditional medical practices is placing high pressures on providing many of such regimens, detecting, and/or treatment remotely from physicians, medical personnel, etc. such as to be able to reduce associated medical costs and/or expenses. It is important to ensure that the treatment and/or regimen remains directed to the condition of the individual such as to potentially provide, or determine not to provide the at least one modified fluid(s) and/or element(s) at least partially in response to the analyzing or detecting the at least one detected fluid(s) and/or element(s). Additionally, it may require considerable time and/or expense of the part of the individual to pursue certain traditional treatments and/or regimens. For these reasons and others, numerous individuals that have been placed on a variety of the treatment and/or regimens may, for one reason or another, fall off their treatment and/or regimen as a result of such factors as the expenses, time associated with treatment, remoteness of treatment, and/or other such associated difficulties. As such, certain embodiments of the detect failure to maintain regimen mechanism 100 can make it easier for the individual to maintain their regimen or treatment as desired, and also monitor the effectiveness of the treatment or regimen.

As such, this disclosure describes a number of embodiments of the detect failure to maintain regimen mechanism 100 that can be configured to analyze an individual; detect an individual as to their compliance to a treatment and/or regimen; and/or treat the individual appropriately depending upon their compliance and/or non-compliance to treatment and/or regimen. Certain embodiments of the detect failure to maintain regimen mechanism can be designed, utilized, modified, and/or provided to suit a particular individual and/or treat for a particular condition. Within this disclosure, the term “condition” can mean, depending upon context: illness, sickness, injury, irregularities, nonconformities, non-compliances, etc.

The detect failure to maintain regimen mechanism 100 can be utilized in variety of ways to allow a physician, a medical technician, and/or another medical care-providing person to provide an adaptive treatment and/or regimen to the individual by utilizing at least partially the feedback mechanism 112 as described with respect to FIG. 1. In certain embodiments, the term “adaptive treatment and/or regimen” may be considered as a treatment and/or regimen that can be altered based at least in part on the detected condition of the individual, the at least one fluid(s) associated with the individual, and/or the at least one element(s) associated with the at least one fluid(s) that is associated with the individual. Within this disclosure, such altering of the treatment or regimen can potentially be performed in the absence of the physician or other medical care-providing person. Such an active treatment and/or regimen may be considered as a treatment and/or regimen having a feedback mechanism that is based at least in part on the detected condition of the individual, the at least one fluid(s) associated with the individual, and/or the at least one element(s) associated with the at least one fluid(s) that is associated with the individual.

Certain embodiments of the treating regimen that can involve the feedback mechanism 112 can, for example, involve applying some treatment (or some level of treatment) to the individual during such treatment periods as when the detected condition of the individual is within some prescribed range, the at least one fluid(s) associated with the individual is within some prescribed range, and/or the at least one element(s) associated with the at least one fluid(s) that is associated with the individual is within some prescribed range. Similarly, the treatment could involve not applying the treatment (or applying a different level of treatment) to the individual during such treatment periods as when the detected condition of the individual is not within some prescribed range, the at least one fluid(s) associated with the individual is not within some prescribed range, and/or the at least one element(s) associated with the at least one fluid(s) that is associated with the individual is not within some prescribed range. These types of treating regimens involving a feedback mechanism can be applied, for example, to apply treatment only in instances to the treatment may benefit the health or condition of the individual.

Certain embodiments of the detect failure to maintain regimen mechanism 100 can also be provided with different levels of automation, operation, and/or control. The different levels of automation, operation, and/or control for certain embodiments of the detect failure to maintain regimen mechanism 100 can vary depending at least partially upon the type of treatment, and/or the complexity of treating the condition, illness, sickness, etc. Certain embodiments of the feedback mechanism can be at least partially computer or controller based, while certain embodiments of the feedback mechanism can rely on input, interaction, or other processes from the individual and/or other humans.

Certain embodiments of the detect failure to maintain regimen mechanism can analyze some aspect of the at least one withdrawn or aspirated fluid(s) and/or element(s), and thereupon allow the user (physician, hospital or ambulance worker, medical technician, etc.) of the detect failure to maintain regimen mechanism 100 to determine the appropriate action based on their knowledge and the analysis. By comparison, certain embodiments of the detect failure to maintain regimen mechanism can be largely automated, such as to be able to automatically inject one or more fluids and/or element(s) based, at least in part, on the analysis of the at least one fluid(s) and/or element(s) (which may be analyzed, controlled, mixed, and/or combined), such that the analysis can also be automated. Automation can be useful on applying a variety of the detect failure to maintain regimen mechanism 100 where the individual and/or phlebotomist is untrained or unskilled.

In certain embodiments, the at least one fluid(s) and/or element(s) that can be withdrawn or aspirated from or injected into the individual may be analyzed to determine some condition or aspect of the individual. Certain embodiments of the detect failure to maintain regimen mechanism 100 can thereby detect whether a condition of the at least one fluid(s) and/or element(s) is outside of some prescribed limit for treatment. In certain embodiments, the detect failure to maintain regimen mechanism 100 can analyze the at least one fluid(s) and/or element(s), and thereby in certain embodiments determine how the at least one fluid(s) and/or element(s) should be modified and thereupon be added to the individual at least partially using the detect failure to maintain regimen mechanism 100. The at least one fluid(s) and/or element(s) that can be contained in a variety of organs can be detected and/or analyzed in a variety of manners to effect treatment therefore.

In certain embodiments, the detect failure to maintain regimen mechanism 100 can modify, filter, and/or replace the at least one fluid(s) and/or element(s), and/or combinations thereof that have been withdrawn or aspirated from the individual. In certain embodiments, the detect failure to maintain regimen mechanism can inject an entirely different fluid(s), element(s), or combination thereof from those withdrawn or aspirated. By comparison, in certain embodiments, the analyzing the at least one fluid(s) and/or element(s) can be used to determine an element that could be added to the at least one fluid(s) and/or element(s) in an effort to benefit the individual at least partially using the detect failure to maintain regimen mechanism 100.

Certain embodiments of the detect failure to maintain regimen mechanism 100, as described with respect to FIG. 1, can include the feedback mechanism 112 that can as described in this disclosure detect the condition of the individual, and/or the at least one fluid(s) and/or element(s) of the individual. In this manner, certain embodiments of the detect failure to maintain regimen mechanism 100 can be configured to provide a suitable treatment or regimen of treatment at least partially in response to the detected condition. Certain embodiments of the feedback mechanism 112 can operate substantially automatically, such as to require limited or no input from the individual and/or person operating or administering the detect failure to maintain regimen mechanism 100. Certain embodiments of the feedback mechanism 112, by comparison, can operate based at least in part upon user input (e.g., the input at least partially derived from the individual operating or administering the detect failure to maintain regimen mechanism 100).

Certain embodiments of the feedback mechanism 112 can thereby be configured to provide a treatment and/or regimen that is at least partially responsive to the detected condition of the individual, the at least one fluid(s) and/or element(s). As such, certain embodiments of dispensing as at least partially perform by the treating and/or dispensing portion 94 can utilize the feedback mechanism 112 to act at least partially in response to the detecting and/or analyzing as performed by the detecting and/or analyzing portion 92, as described in this disclosure.

Certain embodiments of the detect failure to maintain regimen mechanism 100 can thereby be configurable to detect a condition or concentration of the least one fluid(s) and/or element(s) within the individual. Certain embodiments of the detect failure to maintain regimen mechanism 100 can be configured to maintain one or more levels of one or more element(s) with respect to the at least one fluid(s) and/or element(s) that can be maintained within an individual.

Certain embodiments of the treating and/or dispensing portion 94 of the detect failure to maintain regimen mechanism 100 can be utilized to monitor and/or analyze the at least one fluid(s) and/or element(s), etc. within the individual and the detect failure to maintain regimen mechanism 100. In certain instances, the particular type of the treating and/or dispensing portion 94 can largely dictate for what the detect failure to maintain regimen mechanism 100 can be used. For instance, those embodiments of the treating and/or dispensing portion 94 that include patches may preferably be used to primarily transfer the at least one fluid(s) and/or element(s) from the detect failure to maintain regimen mechanism 100 to the individual.

Certain embodiments of the detect failure to maintain regimen mechanism 100 are configured as an implant, as described with respect to FIGS. 2, 3, and/or 4. Certain embodiments of the detect failure to maintain regimen mechanism 100 can interface with a flow of the at least one fluid(s) and/or element(s) 240 as described in this disclosure, such as within a canal, lumen, artery, vein, airway, gas passage, etc. Certain embodiments of the detect failure to maintain regimen mechanism 100 can include, but is not limited to, a fluid/element inlet port 250, the sensor 115, the detect failure to maintain regimen controller 97, the treating and/or dispensing portion 94, and a fluid/element outlet port 252 (these elements are illustrated schematically, but may correspond to certain ones of the blocks as described with respect to FIG. 1). Certain embodiments of the fluid/element inlet port 250 can be configured to direct at least some of the least one fluid and/or element that is being monitored and/or analyzed to the sensor 115. In certain embodiments, the fluid/element inlet port 250 can be in fluid communication with the fluid/element outlet port 252. In certain embodiments, the fluid/element inlet port 250 and/or the fluid/element outlet port 252 can be configured as a tube as described with respect to FIG. 3, or alternately as a space, such that the sensor 115 and/or the treating and/or dispensing portion 94 can be configured adjacent to an airway, blood vessel, etc.

As described with respect to FIG. 3, the least one fluid(s) and/or element(s) can be configured to flow through the combined fluid/element inlet port 250 and the fluid/element outlet port 252 (or adjacent the sensor 115 and/or the treating and/or dispensing portion 94), wherein the least one fluid(s) and/or element(s) can be monitored, analyzed, and/or modified. In certain embodiments, the treating and/or dispensing portion 94 can be configured to heat or cool the at least one fluid(s) and/or element(s) 240, wherein the modification includes changing the temperature of the at least one element(s). With the embodiment of the detect failure to maintain regimen mechanism 100 as described with respect to FIG. 3, the detecting and/or analyzing operations such as performed with the detecting and/or analyzing portion 92 as described with respect to FIG. 1 could be performed at least partially within the individual. With the embodiment of the detect failure to maintain regimen mechanism 100 as described with respect to FIG. 3, the treating and/or dispensing actions such as performed with the treating and/or dispensing portion 94 as described with respect to FIG. 1 could be performed at least partially within the individual. Certain embodiments of the detect failure to maintain regimen mechanism 100 as described with respect to FIG. 3 could thereby be configured as a unitary device including both the detecting and/or analyzing portion 92 as well as the treating and/or dispensing portion 94.

By comparison, certain embodiments of the detect failure to maintain regimen mechanism 100 as described with respect to FIG. 4 can be configured without the treating and/or dispensing portion 94 as described with respect to FIG. 3, but can instead include a communication portion 350 (e.g., wireless) by which information or data obtained from the sensor 115 can be transmitted external of the individual. Based at least in part on the wireless communication, another embodiment or portion of the detect failure to maintain regimen mechanism 100 (which can be situated within the individual or external to the individual) can be configured to receive the information or data, and thereby suitably treat or maintain a regimen of the at least one fluid(s) and/or element(s) for the individual. Known communication, networking, and/or computer techniques can be applied to one or more of the detect failure to maintain regimen mechanism 100 to provide effective operation. Mote devices, such as are known and are commercially available, could be utilized in a number of embodiments of the detect failure to maintain regimen mechanism 100.

Consider that certain embodiments of the communication portion 350 can transmit information or data, while certain embodiments of the communication portion 350 can receive information or data. As such, certain embodiments of detect failure to maintain regimen mechanism 100 can be configured to transmit information to other devices by which the condition of the individual could be monitored.

Additionally, certain embodiments of the detect failure to maintain regimen mechanism 100 can be configured to receive information or data from other devices which can be used to modify the operation of the detect failure to maintain regimen mechanism 100. Certain embodiments of the detect failure to maintain regimen mechanism 100 can include both the communication portion 350 as described with respect to FIG. 4, and the treating and/or dispensing portion 94 as described with respect to FIG. 3. Additionally, certain embodiments of the detect failure to maintain regimen mechanism 100 can include any combination of one or more of the sensor 115, the treating and/or dispensing portion 94, and/or the communication portion 350.

A number of the detect failure to maintain regimen mechanisms 100 may be included in the individual. As such, these mechanisms 100 can communicate with each other throughout different portions of the individual. Such information may be useful in detecting a condition of the at least one fluid(s) and/or element(s), and/or suitable treatments or regimens throughout the individual. Mote and other miniaturized or other semiconductor-based devices could be utilized in certain embodiments of the detect failure to maintain regimen mechanism 100 which are to be implanted into such individuals as humans or animals.

Certain embodiments or portions of the detect failure to maintain regimen controller 97 and/or the feedback mechanism 112, as described with respect to FIG. 1, could be situated internally to the individual such as being integrated within or close to the detect failure to maintain regimen mechanism 100, and thereby does not require any input from the individual, physician, or other medical personnel to provide feedback for normal operation. Such lack of control over the detect failure to maintain regimen controller 97 and/or the feedback mechanism 112 during normal operation of certain embodiments of the detect failure to maintain regimen mechanism 100 may be analogized to a typical pacemaker, in which the care provided may be urgent or difficult to apply, such as to justify internal control. For example, if the detect failure to maintain regimen mechanism 100 is configured to maintain a regimen for dangerous or commonly fatal diseases, and it is difficult to monitor the individual, then in certain embodiments the detect failure to maintain regimen mechanism 100 could be ingested, injected, or otherwise applied to the individual, and the individual would not have to be further concerned about maintaining the regimen or treatment as long as the detect failure to maintain regimen mechanism 100 survives and is operating properly. Certain embodiments of the detect failure to maintain regimen mechanism 100 can be coated with, formed from, or otherwise include an anti-digestive material such as which might increase the duration of the detect failure to maintain regimen mechanism 100 within a desired location of the gastrointestinal tract or other location of the individual.

Certain embodiments or portions of the detect failure to maintain regimen controller 97 and/or the feedback mechanism 112, as described with respect to FIG. 1, while still being internal to the individual such as being integrated within or close to the detect failure to maintain regimen mechanism 100, may provide information or data that can be monitored by the individual, physician, or other medical personnel. Such information and/or data can provide feedback for normal operation, and may be monitored from a remote portion of the detect failure to maintain regimen controller 97 and/or the feedback mechanism 112. For instance, certain embodiments of the detect failure to maintain regimen controller 97 and/or the feedback mechanism 112 can transmit information or data external to the individual indicating to the individual or associated medical personnel the condition of the individual, their fluid(s) and/or their element(s); as well as in certain embodiments the operation of the detect failure to maintain regimen mechanism 100. Based on the information or data that has been received externally of the individual, the operation of the detect failure to maintain regimen mechanism 100 can be monitored or analyzed.

Certain embodiments or portions of the detect failure to maintain regimen controller 97 and/or the feedback mechanism 112, as described with respect to FIG. 1, could be external to the individual, and thereby may provide information or data that can be monitored by the individual, physician, or other medical personnel. Such information and/or data can provide feedback for normal operation, and may be monitored from a remote portion of the detect failure to maintain regimen controller 97 and/or the feedback mechanism 112 to derive a suitable treatment or regimen outside of the individual. In certain embodiments, the derived treatment or regimen could thereupon be applied using certain embodiments of injection or application devices (e.g., pills, needles, non-invasive capsule injections through the skin, other injections, patches, injection ports, tubular applications, etc.). For instance, certain embodiments of the detect failure to maintain regimen controller 97 and/or the feedback mechanism 112 can transmit sensed information or data (e.g., from the sensor) external to the individual indicating to the individual or associated medical personnel the condition of the individual, their fluid(s) and/or their element(s); as well as in certain embodiments the operation of the detect failure to maintain regimen mechanism 100. Based on the information or data that has been received externally of the individual, the operation of the detect failure to maintain regimen mechanism 100 can be monitored or analyzed or other injection or application devices could be utilized to treat the individual, or maintain the regimen for the individual.

These and other embodiments of the detect failure to maintain regimen mechanism 100 can thereby be configured to ensure the individual's regimen is being followed, based at least in part on the condition of the individual, or the condition of the individual's fluid(s) and/or element(s) as sensed by the sensor 115 as situated within the individual, such as within those embodiments of the detect failure to maintain regimen mechanism 100 that can be implanted, consumed or applied orally or through another orifice, applied using other low-invasive techniques, etc.

The embodiment of the detect failure to maintain regimen mechanism 100 as described with respect to FIG. 3 includes the treating and/or dispensing portion 94 that is internal to the individual, and thereby may not utilize invasive devices to treat the individual and/or maintain their regimen. Other embodiments of the detect failure to maintain regimen mechanism thereby can be configured to include an external treating and/or dispensing portion 94 (or an external embodiment of the detect failure to maintain regimen mechanism 100) that can be configured to maintain the regimen of the individual (such as by treating the individual), when suitable.

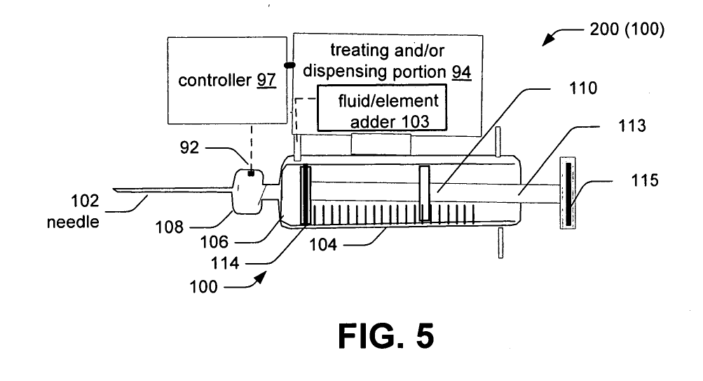

A number of embodiments of external detect failure to maintain regimen mechanisms 100 are now described with respect to FIGS. 2 to 7 that can interface with, for example, certain embodiments of the internal detect failure to maintain regimen mechanisms 100 as described with respect to in FIGS. 1 to 7. In certain instances, certain embodiments of the treating and/or dispensing portion 94 can be configured as to include one or more of a needle or injector 102 embodiment of the detect failure to maintain regimen mechanism 100, an implant 300 embodiment of the detect failure to maintain regimen mechanism 100, a patch 500 embodiment of the detect failure to maintain regimen mechanism 100, a tube 510 embodiment of the detect failure to maintain regimen mechanism 100, and/or any other device that can transfer the at least one fluid(s) and/or element(s), etc. from the detect failure to maintain regimen mechanism 100 to the individual. Certain embodiments of the detect failure to maintain regimen mechanism 100, as described with respect to FIG. 1, can be configured as the treating and/or dispensing portion 94 of the detect failure to maintain regimen mechanism 100 as described with respect to FIG. 5. The embodiments of the detect failure to maintain regimen mechanism 100 (which may be a needle-based embodiment of the treating and/or dispensing portion 94 of the detect failure to maintain regimen mechanism 100), as described with respect to FIG. 5, can include the treating and/or dispensing portion 94 that can be utilized to transfer such fluid(s) as liquid, perhaps mixed with at least one element(s).

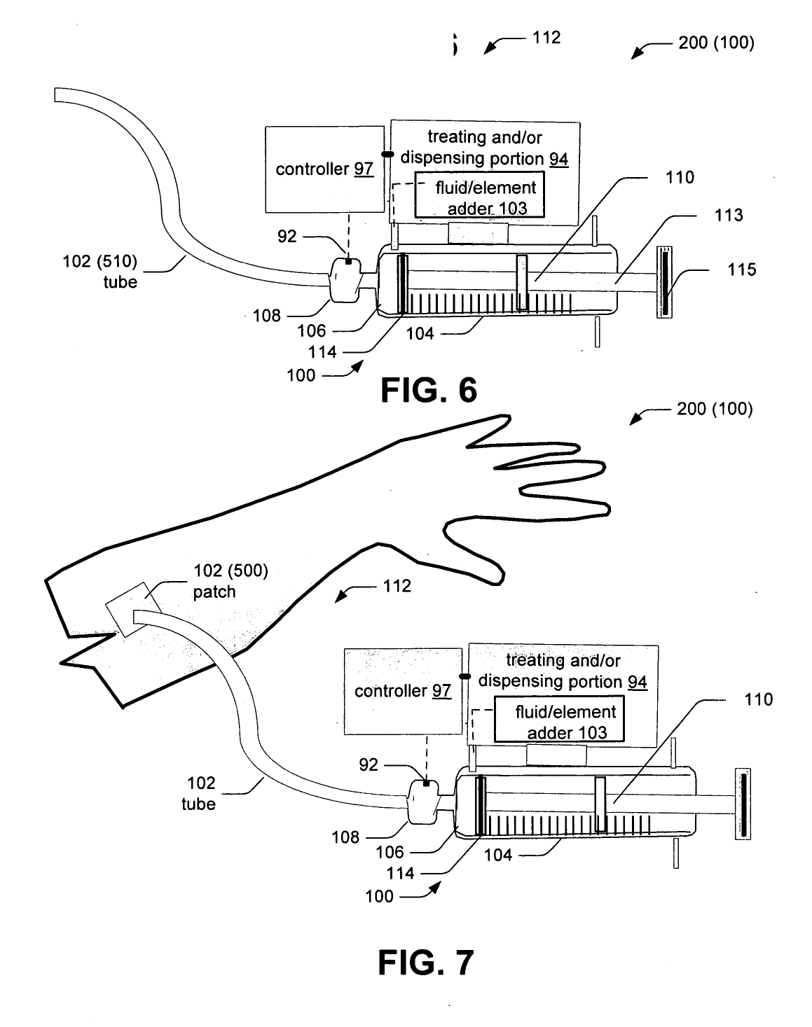

The embodiments of the detect failure to maintain regimen mechanism 100 as described with respect to FIGS. 6 and 7, can include a tubular section that could be utilized to transfer such fluid(s) as gasses, perhaps mixed with at least one element(s) as well as liquids. Certain embodiments of the treating and/or dispensing portion 94 of the detect failure to maintain regimen mechanism 100 can include, but is not limited to, a treating and/or dispensing portion 94, a fluid/element adder 103, a barrel 104, a proximal chamber 106, the detect failure to maintain regimen controller 97, and/or a sheath 108 in fluid communication with the barrel, and/or a plunger 110. Certain embodiments of the plunger 110 can include but are not limited to, e.g., a grip 115, a plunger rod 113, and a seal 114, the seal configurable to form a seal with the proximal chamber 106. In certain embodiments, the treating and/or dispensing portion 94 can be secured to the barrel 104, and can be in fluid communication therewith. In certain embodiments, the transfer can be in the direction from the individual to the detect failure to maintain regimen mechanism 100. In other embodiments, the transfer can be made in the direction from the detect failure to maintain regimen mechanism 100 to the individual. In certain embodiments, the at least one fluid(s) and/or element(s) can be configured primarily as a liquid; in other embodiments the at least one fluid(s) and/or element(s) can be configured primarily as a gas. The particular configuration of the at least one fluid(s) and/or element(s) can in certain instances affect the shape, size, configuration, or other design aspect of the treating and/or dispensing portion 94.

Certain embodiments of the fluid/element adder 103 of the detect failure to maintain regimen mechanism 100 can be configured to add the at least one fluids, elements, components, chemicals, derivatives, heat, coolness, etc. to the at least one fluid(s) and/or element(s) that have been withdrawn or aspirated from the individual using the detect failure to maintain regimen mechanism. Thereupon, in certain embodiments, the at least one fluid(s) and/or element(s) obtained from the fluid/element adder 103 can be combined with the at least one fluid(s) and/or element(s) that have been obtained from the individual to form at least one modified fluid and/or element that can be injected into the individual to afford certain embodiments of the treatment and/or regimen. Other embodiments of the fluid/element adder 103 of the detect failure to maintain regimen mechanism can be configured to add one more fluids, elements, components, chemicals, derivatives, heat, coolness, etc. to the individual based at least in part on detecting of the condition of the individual (or their fluid(s) and/or element(s)) to afford certain embodiments of the treatment. As such, certain embodiments of the fluid/element adder 103 can be configured differently depending upon whether the at least one fluid(s) and/or element(s) being added to the individual is to be combined with the at least one withdrawn or aspirated fluid(s) and/or element(s) from the individual, or whether the at least one fluid(s) and/or element(s) is to be directly applied to the individual, or combined with some other at least one fluid(s) and/or element(s) to yield the at least one modified fluid(s) and/or element(s).

Certain embodiments of the barrel 104 of the detect failure to maintain regimen mechanism 100 as described with respect to FIGS. 5, 6, and 7 can be configured to contain maintain, mix, process, and/or perform other operations on the at least one fluid(s) and/or element(s) that either been withdrawn or aspirated from the individual and/or that have been provided at least in part by the fluid/element adder 103 as described in this disclosure. The configuration and/or usage of certain embodiments of the barrel 104 are generally understood as providing an internal-sliding contact with the plunger 110 to at least partially define the proximal chamber 106 (which can change in dimension as a result of motion of the plunger), and will not be described further in this disclosure.

Certain embodiments of the proximal chamber 106 and/or the sheath 108 of the detect failure to maintain regimen mechanism 100 as described with respect to FIG. 1 can be configured with the detect failure to maintain regimen controller 97. For example, the detect failure to maintain regimen controller 97 can be configured to analyze the at least one fluids and/or element(s) that have been withdrawn from the individual with the detect failure to maintain regimen mechanism. Thereupon, depending upon the analysis, certain embodiments of the fluid/element adder 103 can add the at least one added fluid(s) and/or element(s) to the at least one withdrawn fluids and/or elements to yield the at least one modified fluids and/or elements. In certain embodiments, the at least one modified fluids and/or elements can be combined and/or mixed utilizing some combining or mixing mechanism that is not illustrated but is generally understood, e.g., a mixer of a suitable configuration situated within the barrel 104. In certain embodiments, the at least one modified fluids and/or elements can be injected back into the individual.

Certain embodiments of the detect failure to maintain regimen controller 97 of the detect failure to maintain regimen mechanism 100 as described with respect to FIGS. 1, 2, 3, 4, 5, 6, and 7 can be configured to analyze and/or detect the at least one fluid(s) and/or element(s) contained within the barrel 104. Such detecting and/or analyzing can be performed on the at least one fluid(s) and/or element(s) as withdrawn or aspirated from the individual, as provided by the fluid/element adder 103 or alternately as any combination thereof.

Certain embodiments of the sheath 108 of the detect failure to maintain regimen mechanism 100 can be in fluid communication with at least a part of the barrel, as described with respect to FIGS. 5, 6, and/or 7. Certain embodiments of the sheath 108 can be configured to assist in the apply, withdrawal, aspiration, and/or injection with the detect failure to maintain regimen mechanism 100. For instance, certain embodiments of the sheath 108 can be configured to decrease dead space for air to form within the detect failure to maintain regimen mechanism, thereby limiting the possibility of a transmission of air bubbles to within the blood stream of the individual (the existence of the dead space might, e.g., create an embolism, an inconsistent mixture, or another potentially dangerous or undesirable situation). Certain embodiments of the sheath 108 can also be configured as to limit the use of needle-reuse. The configuration and use of a number of embodiments of the sheath 108 are generally understood, and will not be described further in this disclosure.

Certain embodiments of the plunger 110 of the detect failure to maintain regimen mechanism 100 as described with respect to FIGS. 5, 6, and/or 7 can be configured to, upon actuation and/or displacement, aspirate, inject, and/or withdraw the at least one fluid(s) and/or element(s) between the detect failure to maintain regimen mechanism 100 and the individual. Certain embodiments of the plunger 110 can be configured with a seal that can limit leakage of the at least one fluid(s) and/or element(s) between the plunger 110 and the barrel 104. As such, in certain embodiments, the plunger 110 in combination with the barrel 104 can at least partially form an enclosure for the at least one fluid(s) and/or element(s), wherein the at least one fluid(s) and/or element(s) can escape via the treating and/or dispensing portion 94 upon suitable actuation of the plunger 110 (e.g. by the individual 120 and/or the phlebotomist).

Certain embodiments of the detect failure to maintain regimen controller 97 as described with respect to FIGS. 1 to 7 can be utilized to sense, analyze, and/or detect the at least one fluid(s) and/or element(s) that can be withdrawn or aspirated from the individual at least partially by the detect failure to maintain regimen mechanism 100. Certain embodiments of the treating and/or dispensing portion 94 as described with respect to FIG. 5 can include, for example, a needle 102 that can be configured to withdraw, aspirate, and/or inject, the at least one fluid(s) and/or element(s) that can include, but is not limited to: a liquid, blood, saline, water, a combination of fluids, etc. In certain embodiments, the at least one fluid(s) and/or element(s) can be removed from different locations of the body of the individual. For example, certain embodiments of the detect failure to maintain regimen mechanism 100 can withdraw blood from and/or inject blood into the individual's heart, the individual's aorta, the individual's arteries, the individual's veins, and/or certain of the individual's organs or capillaries. Certain aspects of the withdrawn or aspirated fluid(s) and/or element(s) such as blood may be dependent upon the location where the at least one fluid(s) and/or element(s) was withdrawn or aspirated. For example, oxygenated blood may be desired to be withdrawn or aspirated from, and/or could also be returned to the arteries, the aorta, certain portions of the heart, etc.; while de-oxygenated blood may be desired to be withdrawn or aspirated from, and could be returned to veins, etc.

The sophistication and/or complexity of the detect failure to maintain regimen mechanism can, in many aspects, relate to certain aspects of the detect failure to maintain regimen controller 97. Certain embodiments of the detect failure to maintain regimen mechanism 100 can perform one or more of the following operations entirely within a clean or sterile environment: withdrawing or aspirating the at least one fluid(s) and/or element(s) from the individual 120, analyzing the at least one fluid(s) and/or element(s), combining the at least one fluid(s) and/or element(s) with one or more fluid(s) and/or element(s) to form at least one modified fluid(s) and/or element(s), and/or injecting the at least one modified fluid(s) and/or element(s) into the individual 120. One aspect of the detect failure to maintain regimen mechanism is that the at least one fluid(s) and/or element(s), as well as the at least one modified fluid(s) and/or element(s), can be maintained in a clean or sterilized condition within the detect failure to maintain regimen mechanism during this process.

A variety of applications for the detect failure to maintain regimen mechanism 100 may be provided. For instance, FIG. 5 shows one application of the detect failure to maintain regimen mechanism 100 that includes a needle 102 which can be utilized to withdraw, aspirate, and/or inject liquids blood from the individual, analyze the blood, add suitable elements to the blood to derive modified blood, and return the modified blood to the individual. The embodiment of the treating and/or dispensing portion 94, which can in certain embodiments include the needle, can be used to inject and/or withdraw the at least one fluid(s) and/or element(s) at a variety of locations. For example, certain embodiments of the needle 102 can be injected in arteries, veins, the aorta, the heart, and other embodiments of the body where blood, or other fluid(s) and/or element(s) that can be detected, can be situated or applied. During certain medical treatments or regimens, certain embodiments of the detect failure to maintain regimen mechanism 100 can be applied to a particular suitable or desired location, organ, or system as generally understood in phlebotomy. Thereupon, a desirable quantity of liquid (e.g., blood) can be removed, and the removed liquid can be analyzed to determine which element(s) can be inserted in the fluid(s) and/or element(s).

Upon the combination, mixing, or insertion of the fluid(s) and/or element(s) into the at least one fluid(s) and/or element(s) to produce the at least one modified fluid(s) and/or element(s), the at least one modified fluid(s) and/or element(s) can thereupon be injected using certain embodiments of the detect failure to maintain regimen mechanism 100 back into the individual. In certain embodiments, the site at which the at least one modified fluid(s) and/or element(s) is injected can be the same site at which the at least one fluid(s) and/or element(s) was originally removed (drawn) from, the individual. As such, the number of withdrawal and/or injection sites can be reduced as a result of using one or more sites for one or more processes. Reducing the number of injection sites for a particular treatment and/or regimen can make the treatment and/or regimen easier, less painful, and in certain instances more reliable. Certain regimens, as for cancer, chemotherapy, diabetes, AIDS, etc. rely on a number of injection sites for injections and/or withdrawals of the fluid(s) and/or element(s). In certain instances, certain individuals having low blood pressure, poor blood circulation, etc. may greatly appreciate the reduced number of injection sites. Additionally, there are a number of individuals who find injections and/or withdrawals to be painful or even make them queasy, who will appreciate the reduction of the number of injection sites by certain embodiments of the detect failure to maintain regimen mechanism particularly desirable.

Another additional aspect of the detect failure to maintain regimen mechanism 100 is that it can perform one or more of the operations (e.g., withdrawing, adding to, mixing, injecting) entirely within a clean or sterile environment as afforded by the detect failure to maintain regimen mechanism. For example, the detect failure to maintain regimen mechanism can withdraw or aspirate the at least one fluid(s) and/or element(s) from the individual 120, analyze the at least one fluid(s) and/or element(s), combine the at least one fluid(s) and/or element(s) with one more fluid(s) and/or element(s) to form the at least one modified fluid(s) and/or element(s), and/or inject the at least one modified fluid(s) and/or element(s) into the individual 120 in a manner that can utilize only one injection shot or aperture from the detect failure to maintain regimen mechanism while not allowing impurities to enter the at least one fluid(s) and/or element(s). Considering the large number of injection-needle apertures certain medical treatments and/or regimens can require can make a number of such traditional treatments or regimens difficult, painful, potentially dangerous, and/or time consuming. By comparison, by certain embodiments of the detect failure to maintain regimen mechanism reducing the number of needle shots to provide the treatment and/or regimen can represent an improvement of time, pain, or danger to the individual as well as the medical care provider (e.g., the phlebotomist). As such, by controlling and/or maintaining certain aspects of the environment of certain embodiments of the detect failure to maintain regimen mechanism, the environment that certain fluid(s) and/or element(s) being withdrawn, aspirating, and/or injected using the detect failure to maintain regimen mechanism can be controlled and/or maintained as well.

Certain embodiments of the detect failure to maintain regimen mechanism 100 may be included with a tube 510 as the treating and/or dispensing portion 94 that can be used to remove and/or inject gases such as air, gaseous medication, gaseous anesthesia, other gasses, and/or combinations of gasses as described with respect to FIG. 7. Certain embodiments of the tube 510 can be configured to transfer the at least one fluid(s) and/or element(s), for example, the tube 510 can be configured to pass or extend into the individual's mouth, nasal passage, an incision in the throat, etc. to allow the transfer of gasses between the individual and the detect failure to maintain regimen mechanism. As such, certain embodiments of the treating and/or dispensing portion 94 can include, for example, the tube 510 that can be configured to withdraw, aspirate, or inject a gas such as air, oxygen, gaseous anesthesia, gaseous medications, a combination of gases, etc.

Certain embodiments of the treating and/or dispensing portion 94 of the detect failure to maintain regimen mechanism 100 can be configured as an implant portion, a patch portion, a low-invasive solid, capsule or liquid injector; and can act, for example, as to receive information or data from the sensor 92 which can be configured to analyze the fluid(s) and/or element(s) such as air, which may be analyzed within or as withdrawn from the individual's throat, mouth, nasal passage, longs, etc., and determining the percentage of carbon dioxide in the air. Thereupon, certain embodiments of the treating and/or dispensing portion of the detect failure to maintain regimen mechanism 100 can apply oxygen via a suitable pathway such as the mouth, nasal passage, incision in the larynx, lungs, etc. Certain embodiments of the treating and/or dispensing portion 94 of the detect failure to maintain regimen mechanism 100 can be configured to apply oxygen or other gasses to the lungs can be applied to individuals who are in a nursing home, hospital, emergency room, ambulance, etc.

Certain embodiments of one or more of the detect failure to maintain regimen mechanism 100 can remove oxygen-depleted blood from at least one vein, oxygenate the blood, and return the blood to the arteries thereby bypassing the lungs. During a period of lung functional reduction or inoperability, certain embodiments of the detect failure to maintain regimen mechanism 100 could even act to bypass and/or assist the operation of the lungs. With these embodiments of the detect failure to maintain regimen mechanism 100, the analysis of certain gasses (e.g., O₂, CO₂, N, etc.) entering the detect failure to maintain regimen mechanism can be significant in maintaining the blood at suitable gas levels, as well as detecting the condition of the individual and the blood. The sophistication or complexity of the detect failure to maintain regimen mechanism 100 can be configured or designed depending upon its intended use, for example as a temporary life-maintaining system, as a longer-term maintaining system, or as a detecting system remotely-situated from medical care.

Certain embodiments of the detect failure to maintain regimen mechanism 100 may also be used to administer anesthesia. For example, the level of an anesthesia within an individual may be desired to be varied depending upon level of the at least one fluid(s) and/or element(s) of the individual (e.g., carbon dioxide in the individual's lungs, blood, etc.). As such, certain embodiments of the detect failure to maintain regimen mechanism 100 can sense whether the level of a particular one or more of the at least one fluid(s) and/or element(s) falls outside of some prescribed limit. Depending upon the actual detected or analyzed range of such at least one fluid(s) and/or element(s), the at least one modified fluid(s) and/or element(s) being applied to the individual may be varied from what is typically applied. In certain instances, if the actual detected or analyzed range of the at least one fluid(s) and/or element(s) fall within some prescribed (e.g., “normal”) range, then the treating and/or dispensing portion can dispense a normal level (or even simply maintain the condition of the fluid(s) and/or element(s)) of the at least one modified fluid(s) and/or element(s) to that individual to maintain medication and/or anesthesiology to the individual. By comparison, if the actual detected or analyzed range of the at least one fluid(s) and/or element(s) fall outside of the prescribed (e.g., “normal”) range, then the treating and/or dispensing portion can dispense an altered level of the at least one modified fluid(s) and/or element(s) to that individual to provide an altered medication and/or an anesthesia to the individual. As such, certain embodiments of the detect failure to maintain regimen mechanism 100 can be utilized to provide adaptive medication and/or adaptive anesthesia. Such adaptive medication and/or adaptive anesthesia can be controlled and/or regulated utilizing the feedback mechanism as described with respect to FIG. 1. Such adaptive medication and/or anesthesia can be applied whether the individual is conscious, unconscious, or medicated. Certain embodiments of the detect failure to maintain regimen mechanism can thereby provide an active version of the feedback mechanism.

Certain embodiments of the detect failure to maintain regimen mechanism 100 may not actually have to withdraw the at least one fluid(s) and/or element(s) from the individual to yield the at least one modified fluid(s) and/or element(s). For example, certain embodiments of the detect failure to maintain regimen mechanism 100 can involve a variety of sensing and/or analyzing technology for the sensor 115 as described herein, certain ones of which may rely at least in part on wireless communications. Consider the embodiment of the implant-based sensor 92 as described with respect to FIG. 2, which can be configured as the mote, sensor device, controller device, computer-based device, or other mechanism that can be determined the condition of the at least one fluid(s) and/or element(s) within the individual 120. Thereupon, in certain embodiments, if some fluid or element should be injected and/or withdrawn from the individual, a communication can be established from the implant-based sensor 92 to another embodiment of the detect failure to maintain regimen mechanism 100. The communication can thereupon be utilized to indicate whether fluid(s) and/or element(s) should be injected or withdrawn into or from the individual which can be the embodiments of the detect failure to maintain regimen mechanism as described with respect to FIGS. 1 to 7, or other derivatives therefrom. Thereby, certain embodiments of the detect failure to maintain regimen mechanism 100 can include a portion within the individual and another portion outside of the individual which can in combination detect and/or analyze the condition of the at least one fluid(s) and/or element(s), and depending thereupon treat the individual based upon the detected and/or analyzed condition. Based at least in part on the sensed or analyzed results, certain embodiments of the detect failure to maintain regimen mechanism 100 can inject the at least one fluid(s), element(s), medication(s), and/or anesthesia(s) into the individual at least partially utilizing the treating and/or dispensing portion 94, as described in this disclosure.

Certain embodiments of the treating and/or dispensing portion 94 of the detect failure to maintain regimen mechanism 100 can be considered as an inorganic and/or an organic device based upon such factors as the fluid(s) and/or the element(s) being contained, stored, and/or dispensed therefrom, as well as the duration which the fluid(s) and/or the element(s) are likely to be contained there within, and where the implant embodiment of the treating and/or dispensing portion 94 of the detect failure to maintain regimen mechanism 100 may be situated within the individual 120. For example, certain embodiments of the implant embodiment of the treating and/or dispensing portion 94 of the detect failure to maintain regimen mechanism 100 that are designed to be relatively temporary may be configured to be organic, such as to be able to be attacked, degraded, or disintegrated by organisms within the body of the individual. By comparison, certain embodiments of the implant embodiment of the treating and/or dispensing portion 94 of the detect failure to maintain regimen mechanism 100 that are designed to be relatively permanent may be configured as inorganic, such as to have longer device-lifespans since they may have more resistance against being attacked or degraded by organisms within the body of the individual.

As described in this disclosure, certain embodiments of the treating and/or dispensing portion 94 can be configured to dispense the at least one fluid(s) and/or element(s), and/or some combination thereof from the detect failure to maintain regimen mechanism 100 to the individual, as described in this disclosure. In certain embodiments of the detect failure to maintain regimen mechanism 100, the at least one fluid(s) and/or element(s) may be derived from the individual, from the detecting and/or analyzing portion 92 of the detect failure to maintain regimen mechanism 100, from some alternate source, and/or from some combination thereof.

Certain embodiments of the detect failure to maintain regimen mechanism thereby can detect an indication of (e.g., an extinction or other) a failure to maintain the medical treatment and/or a regimen. Certain embodiments, of the detect failure to maintain regimen mechanism 100 can notify someone utilizing established networking, communication, and/or data transfer techniques and/or otherwise take some appropriate action such as to provide an alarm which may be observed by the individual and/or others. Certain embodiments of the detect failure to maintain regimen mechanism 100 can additionally provide a co-delivery of a microorganism stock with a nutrient or other reagent as one embodiment of the at least one fluid(s) and/or element(s). Certain embodiments of the detect failure to maintain regimen mechanism 100 can be tailored to monitor, detect, or manage flora in one or more of the pulmonary tract, the genito-urinary tract, the digestive tract, or some other system.

FIG. 2 shows one embodiment of the implant embodiment of the treating and/or dispensing portion 94 of the detect failure to maintain regimen mechanism 100 that can be configured to detect for or sense a condition of the at least one fluid(s) and/or element(s). The information and/or data obtained from the embodiment of the implant embodiment of the treating and/or dispensing portion 94 of the detect failure to maintain regimen mechanism 100 as described with respect to FIG. 2 can in certain embodiments be transmitted to a remote location. In certain embodiments, the implant embodiment of the treating and/or dispensing portion 94 of the detect failure to maintain regimen mechanism 100 as described with respect to FIG. 2 can be configured to transmit information relating to the at least one fluid(s) and/or element(s) to another location that may be external of the individual. Another embodiment of the detect failure to maintain regimen mechanism 100 can utilize the received information relating to the at least one fluid(s) and/or element(s) to potentially provide some treatment, etc.

FIG. 7 shows one embodiment of the patch embodiment of the treating and/or dispensing portion 94 of the detect failure to maintain regimen mechanism 100 in which certain embodiments can be configured to transfer the at least one fluid(s) and/or element(s) to the individual. Certain embodiments of the patch embodiment of the treating and/or dispensing portion 94 of the detect failure to maintain regimen mechanism 100 can be configured primarily as a dispenser to interact with the detect failure to maintain regimen mechanism 100 to allow the at least one fluid(s) and/or element(s) to flow from the individual perhaps to the patch embodiment of the treating and/or dispensing portion 94 of the detect failure to maintain regimen mechanism 100, and thereby act at least in part as the treating and/or dispensing portion 94 as described with respect to FIG. 1. In certain embodiments, the patch embodiment of the treating and/or dispensing portion 94 of the detect failure to maintain regimen mechanism 100 may be kept in contact with the individual for an extended duration, or alternately may be removed relatively quickly, depending upon the type of treatment and/or regimen. Certain embodiments of the detect failure to maintain regimen mechanism can provide chemicals (e.g., for chemotherapy), radioactive isotopes (e.g., for radiation), light or other electromagnetic radiation (e.g., for laser ablation), or other embodiments of the element.

Certain embodiments of the patch embodiment of the treating and/or dispensing portion 94 of the detect failure to maintain regimen mechanism 100 as described with respect to FIG. 7 can also include the detecting and/or analyzing portion 92 as described with respect to FIG. 1. Certain embodiments of the patch portion can also include the sensor 115 as described with respect to FIG. 1, and thereby act at least in part as the detecting and/or analyzing portion 92 as described with respect to FIG. 1 to sense at least one condition of the individual and/or the condition of the at least one fluid(s) and/or element(s) of the individual. Thereupon, certain embodiments of the treating and/or dispensing portion 94 could dispense a variety of the at least one fluid(s) and/or element(s) (or even dispense nothing) as appropriate based at least in part on the sensed condition of the individual. A variety of patches are commercially available and generally known by those skilled in the technology, and may be utilized as the patch embodiment of the treating and/or dispensing portion 94 of the detect failure to maintain regimen mechanism 100.

FIG. 6 shows another embodiment of the treating and/or dispensing portion 94 of the detect failure to maintain regimen mechanism 100 that can be configured to transfer the at least one fluid(s) and/or element(s) (particularly those configured as gases) between the individual and the detect failure to maintain regimen mechanism. Certain embodiments of the treating and/or dispensing portion 94 of the detect failure to maintain regimen mechanism 100 can be flexible such as to assist insertion, while other embodiments can be relatively rigid. Certain embodiments of the treating and/or dispensing portion 94 of the detect failure to maintain regimen mechanism 100 can be configurable to interface with a variety of the patch embodiment of the treating and/or dispensing portion 94 of the detect failure to maintain regimen mechanism 100 as described with respect to FIG. 7. For example, the patch embodiment of the treating and/or dispensing portion 94 of the detect failure to maintain regimen mechanism 100 can be particularly located at a desired location such as to be able to dispense the at least one fluid(s) and/or element(s), such as might be used to treat the individual. For instance, consider the embodiment where the individual is being treated for throat cancer. Certain embodiments of the treating and/or dispensing portion 94 of the detect failure to maintain regimen mechanism 100 can position certain embodiments of the implant embodiment of the treating and/or dispensing portion 94 of the detect failure to maintain regimen mechanism 100 at a desired location, such as the throat of the individual.

Following implant, a variety of techniques can be provided to derive the relative position of the implant embodiment of the treating and/or dispensing portion 94 of the detect failure to maintain regimen mechanism 100, and thereupon dispense the at least one fluid(s)/element(s) as appropriate for the particular illness or sickness. As such, cancer, heart disease, AIDS, and a variety of other illnesses or diseases can be suitably detected and/or analyzed at the particular location of the illness or disease, and precisely targeted utilizing a number of embodiments of the detect failure to maintain regimen mechanism 100. Certain embodiments of the treating and/or dispensing portion 94 of the detect failure to maintain regimen mechanism 100 can include the tubular segment 102 that can be configured to transfer gaseous or liquid embodiments of the at least one the fluid(s) and/or element(s). For example, certain embodiments of the tubular segment 102 can be inserted down the throat, nasal passage, larynx, etc. of the individual. As such, the embodiment of the treating and/or dispensing portion 94 of the detect failure to maintain regimen mechanism 100, as described with respect to FIG. 6, can be configured to detect and/or analyze gasses.

Certain embodiments of the treating and/or dispensing portion 94 can be configured to dispense the at least one fluid(s) and/or element(s) based at least in part on feedback information derived by the feedback mechanism, as described in this disclosure with respect to FIG. 1. As such, in certain embodiments, analyzed information as derived at least in part from analysis of the at least one fluid(s) and/or element(s) that can be provided as treatment at least in part on the detecting and/or analyzing portion 92.

### 2. Certain Embodiments of the Detecting and/or Treating Controller

This disclosure describes a number of embodiments of the detect failure to maintain regimen controller 97 as described with respect to FIG. 1 that are intended to control operations of the detect failure to maintain regimen mechanism 100. Certain embodiments of the detect failure to maintain regimen controller 97 can act as, and is provided the functionality of, the at least a portion of the feedback mechanism as described with respect to FIG. 1. Certain embodiments of the detect failure to maintain regimen controller 97 can be operationally, functionally, or structurally divided across a number of distinct units which together can act as the detect failure to maintain regimen mechanism 100.

FIG. 1 shows a block diagram of certain embodiments of the detect failure to maintain regimen mechanism 100 that include the detect failure to maintain regimen controller 97. Certain embodiments of the detect failure to maintain regimen mechanism 100 can include, but are not limited to, any particular configuration of the detect failure to maintain regimen controller 97. Certain embodiments of the detect failure to maintain regimen controller 97 can be computer based, mote based, and/or electronics based. Certain embodiments of the detect failure to maintain regimen controller 97 can be segmented into modules, certain of which can be at least partially within the individual's body while others of which can be at least partially outside of the individual's body. Certain embodiments of the detect failure to maintain regimen controller 97 can utilize a variety of wireless communication and/or networking technology to allow information, data, etc. to be transferred to the various distinct portions of the detect failure to maintain regimen mechanism 100.

Certain embodiments of the detect failure to maintain regimen controller 97 can vary as to their automation, complexity, and/or sophistication; and can be utilized to analyze the at least one fluid(s) and/or element(s) withdrawn or aspirated from and/or injected into the individual, control the at least one fluid(s) and/or element(s) added to the individual, and/or control the at least one fluid(s) and/or element(s) withdrawn, aspirated and/or fluid injected relative the individual. As described within this disclosure, multiple ones of the different embodiments of the detect failure to maintain regimen mechanism 100 can transfer information about the individual 120 or their condition, information or data relating to the fluid(s) and/or element(s), signals, etc. via a communication link to or from the remote detecting location or some intermediate device as might be associated with detecting, monitoring, and/or other activities.

Certain embodiments of the detect failure to maintain regimen controller 97, as well as certain embodiments of the detect failure to maintain regimen mechanism 100 in general, can utilize distinct firmware, hardware, and/or software technology. For example, mote-based technology, microprocessor-based technology, microcomputer-based technology, general-purpose computer technology, specific-purpose to computer technology, and a variety of other computer technologies can be utilized for certain embodiments of the detect failure to maintain regimen controller 97, as well as certain embodiments of the detect failure to maintain regimen mechanism 100.

Certain embodiments of the detect failure to maintain regimen controller 97 can include a processor 803 such as a central processing unit (CPU), a memory 807, a circuit or circuit portion 809, and an input output interface (I/O) 811 that may include a bus (not shown). Certain embodiments of the detect failure to maintain regimen controller 97 of the detect failure to maintain regimen mechanism 100 can include and/or be a portion of a general-purpose computer, a specific-purpose computer, a microprocessor, a microcontroller, a personal display assistant (PDA), a cellular phone, a wireless communication device, a hard-wired phone, and/or any other known suitable type of communications device, computer, and/or controller that can be implemented in hardware, software, electromechanical devices, and/or firmware. Certain portions of the detect failure to maintain regimen controller 97 of the detect failure to maintain regimen mechanism 100 can utilize physically or operably configurable phlebotomy, detecting, and/or other associated or unrelated activities. Certain embodiments of the processor 803, as described with respect to FIG. 1, can perform the processing and arithmetic operations for certain embodiments of the detect failure to maintain regimen controller 97 of the detect failure to maintain regimen mechanism 100. For instance, dosing, rate of application, and certain other such aspects can be performed by the processor 803. Certain embodiments of the detect failure to maintain regimen controller 97 of the detect failure to maintain regimen mechanism 100 can control the signal processing, database querying and response, computational, timing, data transfer, and other processes associated with certain embodiments of the detect failure to maintain regimen controller 97 of the detect failure to maintain regimen mechanism 100.

Certain embodiments of the memory 807 of the detect failure to maintain regimen controller 97 can include a random access memory (RAM) and/or read only memory (ROM) that together can store the computer programs, operands, and other parameters that control the operation of certain embodiments of the detect failure to maintain regimen controller 97 of the detect failure to maintain regimen mechanism 100. The memory 807 can be configurable to contain the defibrillation information or individual information obtained, retained, or captured by the detect failure to maintain regimen controller 97 of the detect failure to maintain regimen mechanism 100.

Certain embodiments of the bus can be configurable to provide for digital information transmissions between the processor 803, circuits 809, memory 807, I/O 811, and/or the image memory or storage device (which may be integrated or removable). In this disclosure, the memory 807 can be configurable as RAM, flash memory, semiconductor-based memory, of any other type of memory that can be configurable to store data pertaining to images. The bus also connects I/O 811 to the portions of certain embodiments of the detect failure to maintain regimen controller 97 of the detect failure to maintain regimen mechanism 100 that either receive digital information from, or transmit digital information to other portions of the detect failure to maintain regimen mechanism 100.

Certain embodiments of the detect failure to maintain regimen controller 97 of the detect failure to maintain regimen mechanism 100 as described with respect to FIG. 1 can include a transmitter portion (not shown) that can be either included as a portion of certain embodiments of the detect failure to maintain regimen controller 97 of the detect failure to maintain regimen mechanism 100, or alternately can be provided as a separate unit (e.g., microprocessor-based). In certain embodiments, the transmitter portion can transmit image information between certain embodiments of the detect failure to maintain regimen controller 97 of the detect failure to maintain regimen mechanism 100. As such, certain embodiments of the detect failure to maintain regimen mechanism 100 can utilize network-based or communication based technology as generally known in those arts to provide data or information communications between multiple devices which together can act as the detect failure to maintain regimen mechanism 100.

Certain embodiments of the detect failure to maintain regimen controller 97 of the detect failure to maintain regimen mechanism 100 as described with respect to FIG. 1 can include an operation altering portion (not shown) that can be either included as a portion of certain embodiments of the detect failure to maintain regimen controller 97 of the detect failure to maintain regimen mechanism 100, or alternately can be provided as a separate unit (e.g., microprocessor-based). Examples of operation altering portions include, but are not limited to, altering a resolution, altering a contextual library, altering an aspect ratio, altering a color intensity and/or brightness or particular detect failure to maintain regimen mechanism 100.

Certain embodiments of the memory 807 can provide one example of a memory storage portion. In certain embodiments, the detected or monitored value includes but is not limited to: a percentage of the memory 807, a number of images that are stored in the memory 807, or for data storage or recording interval (audio or video recording intervals).

To provide for overflow ability for the memory 807 of certain embodiments of the detect failure to maintain regimen controller 97 of the detect failure to maintain regimen mechanism 100, the image storage device can be operably coupled to the memory 807 to allow a controllable transmitting of memory data from certain embodiments of the detect failure to maintain regimen controller 97 of the detect failure to maintain regimen mechanism 100 when the detected value of data within the memory 807 (e.g., the memory storage portion) exceeds a prescribed value. The prescribed value can include, e.g., some percentage amount or some actual amount of the value.

In certain embodiments, a secondary communication link can be established between the certain embodiments of the detect failure to maintain regimen controller 97 of the detect failure to maintain regimen mechanism 100. The secondary communication link can be structured similar to as a communication link, or alternatively can utilize network-based computer connections, Internet connections, etc. to provide information and/or data transfer between certain embodiments of the detect failure to maintain regimen controller 97 of the detect failure to maintain regimen mechanism 100. As such, certain embodiments of detect failure to maintain regimen mechanism 100, a remotely-situated doctor, medical technician, nurse, medical analysis, etc. could analyze data or other such information transmitted from the detect failure to maintain regimen mechanism, and thereupon analyze suitable operation of the detect failure to maintain regimen mechanism based at least partially upon the analysis. In certain embodiments, the remotely-situated doctor, medical technician, nurse, medical analysis, etc. could communicate with a phlebotomist or other individual operating the detect failure to maintain regimen mechanism 100.

In certain embodiments, the remotely-situated doctor, medical technician, nurse, medical analysis, etc. could respond to the information of data transmitted from the detect failure to maintain regimen mechanism 100 with remote medical information, that in certain embodiments could somehow be applied to and/or utilized by the detect failure to maintain regimen mechanism 100. For instance, certain embodiments of the remote medical information could include but is not limited to: detect failure to maintain regimen mechanism operational information (e.g., information instructing the phlebotomist to, or not to, inject the contents of the detect failure to maintain regimen mechanism into the individual), element adding instructions (e.g., add the following contents to the contents in the detect failure to maintain regimen mechanism, heat the contents of the detect failure to maintain regimen mechanism, etc.), etc. Certain embodiments of the remote medical information could be largely automated, or alternatively remain primarily up to the phlebotomist to apply. Additionally, a variety of communication devices, networking devices, computing devices, user interfaces, etc. could be utilized to enhance communications between a remotely-situated doctor, medical technician, nurse, medical analysis, etc. and the phlebotomist.

In certain embodiments of the detect failure to maintain regimen controller 97 of the detect failure to maintain regimen mechanism 100, the particular elements of certain embodiments of the detect failure to maintain regimen controller 97 of the detect failure to maintain regimen mechanism 100 (e.g., the processor 803, the memory 807, the circuits 809, and/or the I/O 811) can provide a detecting or monitored function to protect or sense at least one condition of the individual and/or their fluid(s) and/or element(s). A detecting function as provided by certain embodiments of the detect failure to maintain regimen controller 97 of the detect failure to maintain regimen mechanism 100 can be compared to a prescribed limit, such as whether the number of images contained in the memory 807, the amount of data contained within the memory 807, or some other measure relating to the memory is approaching some value. The limits to the value can, in different embodiments, be controlled by the user or the manufacturer of certain embodiments of the detect failure to maintain regimen controller 97 of the detect failure to maintain regimen mechanism 100. In certain embodiments, the memory 807 can store such information as data, information, displayable information, readable text, motion images, video images, and/or audio images, etc.

In certain embodiments, the I/O 811 provides an interface to control the transmissions of digital information between each of the components in certain embodiments of the detect failure to maintain regimen controller 97 of the detect failure to maintain regimen mechanism 100. The I/O 811 also provides an interface between the components of certain embodiments of the detect failure to maintain regimen controller 97 of the detect failure to maintain regimen mechanism 100. The circuits 809 can include such other user interface devices as a display and/or a keyboard.

In other embodiments, the detect failure to maintain regimen controller 97 of the detect failure to maintain regimen mechanism 100 can be, constructed as a specific-purpose computer such as an application-specific integrated circuit (ASIC), a microprocessor, a microcomputer, or other similar devices.

A variety of devices, locations, etc. could be configured as and/or include certain embodiments of the detect failure to maintain regimen mechanism 100. For example, a portion of an automobile or ambulance (either internal or external location thereof) could be provided with certain embodiments of the detect failure to maintain regimen mechanism. A trained dog (such as used in ski patrols, etc.) could be fitted with certain embodiments of the detect failure to maintain regimen mechanism 100, and allowed to run to those in need of emergency medical care. Hospitals, care centers, schools, ski patrols, life guards, workplaces, ambulance workers, forest workers, etc. could each be provided with their individualized version of the detect failure to maintain regimen mechanism 100, as necessary. A variety of vehicles, seats, etc. can also be configured with certain embodiments of the detect failure to maintain regimen mechanism 100. For instance, certain hospitals, rest homes, doctor offices, etc. can be provided with certain embodiments of the detect failure to maintain regimen mechanism 100.

### 3. Certain Embodiments of Detect Failure to Maintain Regimen Mechanism with Relevant Flowcharts

Within the disclosure, flow charts of the type described in this disclosure apply to method steps as performed by a computer or controller. The flow charts can also apply to apparatus devices, such as an antenna or a node associated therewith that can include, e.g., a general-purpose computer or specialized-purpose computer whose structure along with the software, firmware, electromechanical devices, and/or hardware, can perform the process or technique described in the flow chart.

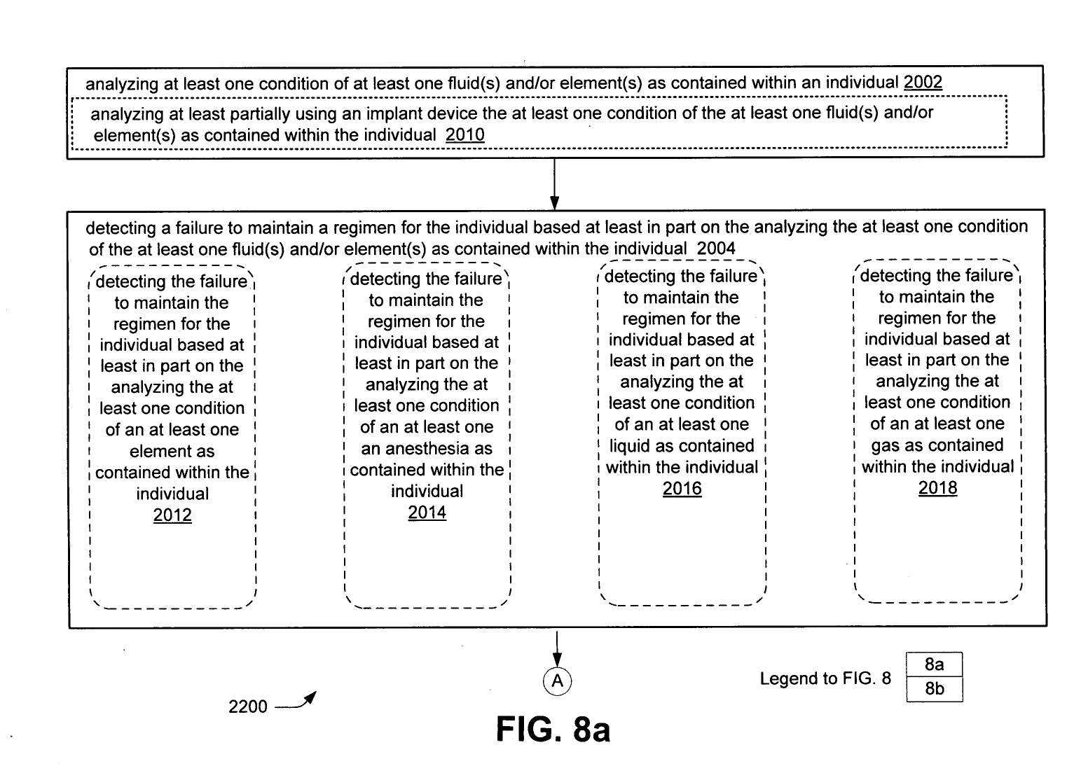

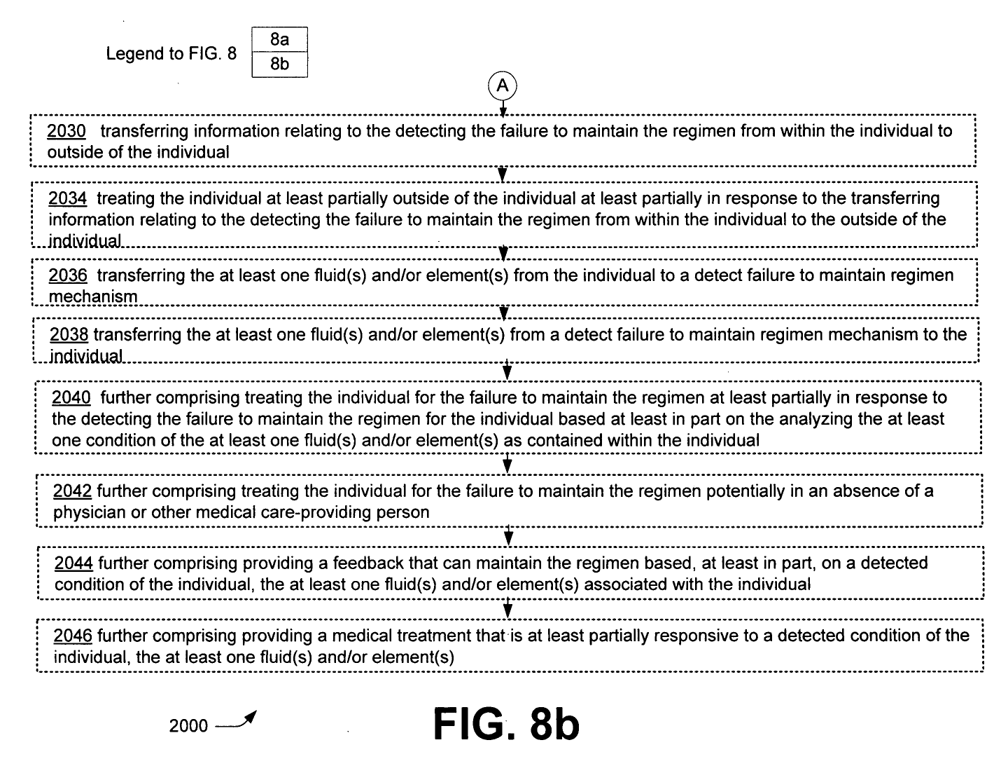

One embodiment of a high-level flowchart of a detect failure to maintain a regimen technique 2000 is described with respect to FIG. 8 (including FIGS. 8a and 8b) can include, but is not limited to, operations 2002 and 2004, and optional operations 2030, 2034, 2036, 2038, 2040, 2042, 2044, and/or 2046. One embodiment of operation 2002 can include, but is not limited to, optional operation 2010. One embodiment of operation 2004 can include, but is not limited to, optional operations 2012, 2014, 2016, and/or 2018. The high-level flowchart of FIG. 8 (including FIGS. 8a and 8b) should be considered in combination with the embodiments of the detect failure to maintain regimen mechanism 100, as described with respect to FIGS. 1 to 7. One embodiment of operation 2002 can include, but is not limited to, analyzing at least one condition of at least one fluid(s) and/or element(s) as contained within an individual. For example, certain embodiments of the detect failure to maintain regimen mechanism 100 as described with respect to FIGS. 1 to 7 can be configured to analyze the condition of the fluid(s) (which may include a gas, a liquid, or any combination thereof) and/or element(s) within the individual. One embodiment of operation 2004 can include, but is not limited to, detecting a failure to maintain a regimen for the individual based at least in part on the analyzing the at least one condition of the at least one fluid(s) and/or element(s) as contained within the individual. For example, detecting a failure to maintain a regimen such as may require modification of the at least one fluid(s) and/or element(s) (or alternatively no modification). One embodiment of the analyzing at least one condition of at least one fluid(s) and/or element(s) as contained within an individual of operation 2002 can include operation 2010, that can include but is not limited to analyzing at least partially using an implant device the at least one condition of the at least one fluid(s) and/or element(s) as contained within the individual. For example, analyzing using the implant embodiment of the detect failure to maintain regimen mechanism 100 as described with respect to FIG. 2, 3, or 4. One embodiment of the detecting a failure to maintain a regimen for the individual based at least in part on the analyzing the at least one condition of the at least one fluid(s) and/or element(s) as contained within the individual of operation 2004 can include operation 2012, that can include but is not limited to detecting the failure to maintain the regimen for the individual based at least in part on the analyzing the at least one condition of an at least one element as contained within the individual. For example, the detecting the failure to maintain the regimen can be performed at least partially in response to the analyzing the condition of the at least one element as contained in the individual. One embodiment of the detecting a failure to maintain a regimen for the individual based at least in part on the analyzing the at least one condition of the at least one fluid(s) and/or element(s) as contained within the individual of operation 2004 can include operation 2014, that can include but is not limited to detecting the failure to maintain the regimen for the individual based at least in part on the analyzing the at least one condition of an at least one an anesthesia as contained within the individual. For example, the detecting the failure to maintain the regimen is at least partially a measure of the anesthesia of the individual. One embodiment of the detecting a failure to maintain a regimen for the individual based at least in part on the analyzing the at least one condition of the at least one fluid(s) and/or element(s) as contained within the individual of operation 2004 can include operation 2016, that can include but is not limited to detecting the failure to maintain the regimen for the individual based at least in part on the analyzing the at least one condition of an at least one liquid as contained within the individual. For example, the detecting the failure to maintain the regimen can be based at least in part on analyzing the condition of an at least one liquid. One embodiment of the detecting a failure to maintain a regimen for the individual based at least in part on the analyzing the at least one condition of the at least one fluid(s) and/or element(s) as contained within the individual of operation 2004 can include operation 2018, that can include but is not limited to detecting the failure to maintain the regimen for the individual based at least in part on the analyzing the at least one condition of an at least one gas as contained within the individual. For example, certain embodiments of the detect failure to maintain regimen mechanism 100 can analyze at least one gas. One embodiment of operation 2030 can include but is not limited to, transferring information relating to the detecting the failure to maintain the regimen from within the individual to outside of the individual. For example, transferring information relating from the detecting the failure to maintain the regimen from within the individual (e.g., a lumen of the individual) to outside of the individual, such as between multiple detect failure to maintain regimen mechanisms. One embodiment of operation 2034 can include but is not limited to, treating the individual at least partially outside of the individual at least partially in response to the transferring information relating to the detecting the failure to maintain the regimen from within the individual to the outside of the individual. For example, treating the individual at least partially outside of the individual such as by determining fluid(s) and/or element(s) that should be added to the individual. One embodiment of operation 2036 can include but is not limited to, transferring the at least one fluid(s) and/or element(s) from the individual to a detect failure to maintain regimen mechanism. For example, transferring the at least one fluid(s) and/or element(s) from the individual to a detect failure to maintain regimen mechanism, such as might be situated outside of the individual. One embodiment of operation 2038 can include but is not limited to, transferring the at least one fluid(s) and/or element(s) from a detect failure to maintain regimen mechanism to the individual. For example, transferring the at least one fluid(s) and/or element(s), such as might be modified and/or different from, certain embodiments of the at least one fluid(s) and/or element(s) within the individual. One embodiment of operation 2040 can include but is not limited to, further comprising treating the individual for the failure to maintain the regimen at least partially in response to the detecting the failure to maintain the regimen for the individual based at least in part on the analyzing the at least one condition of the at least one fluid(s) and/or element(s) as contained within the individual. For example, analyzing the condition of the at least one fluid(s) and/or element(s) as contained within the individual, such as with an implant device or a syringe device having a tube. One embodiment of operation 2042 can include but is not limited to, further comprising treating the individual for the failure to maintain the regimen potentially in an absence of a physician or other medical care-providing person. For example, using certain embodiments of the detect failure to maintain regimen mechanism 100 as described with respect to FIGS. 1 to 7 in the absence of a doctor or medical personnel. One embodiment of operation 2044 can include but is not limited to, further comprising providing a feedback that can maintain the regimen based, at least in part, on a detected condition of the individual, the at least one fluid(s) and/or element(s) associated with the individual. For example, using a feedback mechanism as described with respect to FIG. 1. One embodiment of operation 2046 can include but is not limited to, further comprising providing a medical treatment that is at least partially responsive to a detected condition of the individual, the at least one fluid(s) and/or element(s). For example, providing a medical treatment that is at least partially responsive to the condition of the individual. The order of the operations, methods, mechanisms, etc. as described with respect to FIG. 8 (including FIGS. 8a and 8b) is intended to be illustrative in nature, and not limited in scope.

In one or more various aspects, related systems include but are not limited to circuitry and/or programming for effecting the herein-referenced method aspects; the circuitry and/or programming can be virtually any combination of hardware, software, electromechanical system, and/or firmware configurable to effect the herein-referenced method aspects depending upon the design choices of the system designer.

### 4. Conclusion

This disclosure provides a number of embodiments of the detect failure to maintain regimen mechanism. The embodiments of the detect failure to maintain regimen mechanism as described with respect to this disclosure are intended to be illustrative in nature, and are not limiting its scope.

Those having skill in the art will recognize that the state of the art has progressed to the point where there is little distinction left between hardware, firmware, and/or software implementations of aspects of systems, such as may be utilized in the detect failure to maintain regimen mechanism. The use of hardware, firmware, and/or software can therefore generally represent (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle can vary with the context in which the processes and/or systems and/or other technologies are deployed. For example, if an implementer determines that speed and accuracy are paramount, the implementer and/or designer of the detect failure to maintain regimen mechanism may opt for mainly a hardware and/or firmware vehicle. In alternate embodiments, if flexibility is paramount, the implementer and/or designer may opt for mainly a software implementation. In yet other embodiments, the implementer and/or designer may opt for some combination of hardware, software, and/or firmware. Hence, there are several possible techniques by which the processes and/or devices and/or other technologies described herein may be effected, none of which is inherently superior to the other in that any vehicle to be utilized is a choice dependent upon the context in which the vehicle can be deployed and the specific concerns (e.g., speed, flexibility, or predictability) of the implementer, any of which may vary.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), motes, Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory; and transmission type media such as digital and analog communication links using TDM or IP based communication links (e.g., packet links).

All of the above U.S. patents, U.S. patent application publications, U.S. patent applications, foreign patents, foreign patent applications and non-patent publications referred to in this specification and/or listed in any Application Data Sheet, are incorporated herein by reference, in their entireties.

The herein described disclosure can envision combinations of different elements that can be combined with the at least one fluid(s) and/or element(s), and thereupon injected into the individuals. Additionally, combinations of the at least one fluid(s) and/or element(s) can be withdrawn, aspirated, and/or injected. For example, certain blood transfusion patients may have their own blood removed, detecting, and depending on the condition or analysis of the blood, additional blood may be combined with the analyzed blood to at least partially form the at least one modified fluid(s) and/or element(s) that can be injected or transfused back into the individual. Such depicted architectures of the detect failure to maintain regimen mechanism are merely exemplary, and that in fact many other architectures can be implemented which achieve the same functionality. In a conceptual sense, certain mixtures or combinations of the at least one fluid(s) and/or element(s) to achieve the same functionality can be effectively “associated” such that the desired functionality is achieved. Hence, any combination of fluid(s) and/or element(s) to achieve a particular functionality can be seen as “associated with” each other such that the desired functionality is achieved, irrespective of mixtures or intermedial fluids.

It is to be understood by those skilled in the art that, in general, that the terms used in the disclosure, including the drawings and the appended claims (and especially as used in the bodies of the appended claims), are generally intended as “open” terms. For example, the term “including” should be interpreted as “including but not limited to”; the term “having” should be interpreted as “having at least”; and the term “includes” should be interpreted as “includes, but is not limited to”; etc. In this disclosure and the appended claims, the terms “a”, “the”, and “at least one” positioned prior to one or more goods, items, and/or services are intended to apply inclusively to either one or a plurality of those goods, items, and/or services.

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that could have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that could have A alone, B alone, C alone, A and B together, A and C together, B and C together, and/or A, B, and C together, etc.).

Those skilled in the art will appreciate that the herein-described specific exemplary processes and/or devices and/or technologies are representative of more general processes and/or devices and/or technologies taught elsewhere herein, such as in the claims filed herewith and/or elsewhere in the present application.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method comprising:
analyzing at least one condition of at least one fluid(s) and/or element(s) as contained within an individual; and
detecting a failure to maintain a regimen for the individual based at least in part on the analyzing the at least one condition of the at least one fluid(s) and/or element(s) as contained within the individual.

2. The method of claim 1, wherein the analyzing at least one condition of at least one fluid(s) and/or element(s) as contained within an individual comprises:
analyzing at least partially using an implant device the at least one condition of the at least one fluid(s) and/or element(s) as contained within the individual.

3. The method of claim 1, wherein the detecting a failure to maintain a regimen for the individual based at least in part on the analyzing the at least one condition of the at least one fluid(s) and/or element(s) as contained within the individual comprises:
detecting the failure to maintain the regimen for the individual based at least in part on the analyzing the at least one condition of an at least one element as contained within the individual.

4. The method of claim 1, wherein the detecting a failure to maintain a regimen for the individual based at least in part on the analyzing the at least one condition of the at least one fluid(s) and/or element(s) as contained within the individual comprises:
detecting the failure to maintain the regimen for the individual based at least in part on the analyzing the at least one condition of an at least one an anesthesia as contained within the individual.

5. The method of claim 1, wherein the detecting a failure to maintain a regimen for the individual based at least in part on the analyzing the at least one condition of the at least one fluid(s) and/or element(s) as contained within the individual comprises:
detecting the failure to maintain the regimen for the individual based at least in part on the analyzing the at least one condition of an at least one liquid as contained within the individual.

6. The method of claim 1, wherein the detecting a failure to maintain a regimen for the individual based at least in part on the analyzing the at least one condition of the at least one fluid(s) and/or element(s) as contained within the individual comprises:
detecting the failure to maintain the regimen for the individual based at least in part on the analyzing the at least one condition of an at least one gas as contained within the individual.

7. The method of claim 1, further comprising:
transferring information relating to the detecting the failure to maintain the regimen from within the individual to outside of the individual.

8. The method of claim 1, further comprising:
transferring information relating to the detecting the failure to maintain the regimen from within the individual to outside of the individual; and
treating the individual at least partially outside of the individual at least partially in response to the transferring information relating to the detecting the failure to maintain the regimen from within the individual to the outside of the individual.

9. The method of claim 1, further comprising:
transferring the at least one fluid(s) and/or element(s) from the individual to a detect failure to maintain regimen mechanism.

10. The method of claim 1, further comprising:
transferring the at least one fluid(s) and/or element(s) from a detect failure to maintain regimen mechanism to the individual.

11. The method of claim 1, further comprising treating the individual for the failure to maintain the regimen at least partially in response to the detecting the failure to maintain the regimen for the individual based at least in part on the analyzing the at least one condition of the at least one fluid(s) and/or element(s) as contained within the individual.

12. The method of claim 1, further comprising treating the individual for the failure to maintain the regimen potentially in an absence of a physician or other medical care-providing person.

13. The method of claim 1, further comprising providing a feedback that can maintain the regimen based, at least in part, on a detected condition of the individual, the at least one fluid(s) and/or element(s) associated with the individual.

14. The method of claim 1, further comprising providing a medical treatment that is at least partially responsive to a detected condition of the individual, the at least one fluid(s) and/or element(s).

15. An apparatus, comprising:
a detect failure to maintain regimen mechanism that is configurable to detect a failure to maintain a medical regimen by an individual.

16. The apparatus of claim 15, further comprising:
the detect failure to maintain regimen mechanism configurable to treat the individual.

17. The apparatus of claim 15, further comprising:
the detect failure to maintain regimen mechanism configurable to notify a person as to the failure to maintain at least partially in response to the detect the failure to maintain the medical regimen by the individual.

18. The apparatus of claim 15, further comprising:
a feedback mechanism configurable to detect failure to maintain regimen mechanism.

19. The apparatus of claim 15, further comprising:
the detect failure to maintain regimen mechanism configurable to co-deliver a microorganism stock with a nutrient or other reagent.

20. The apparatus of claim 15, further comprising:
the detect failure to maintain regimen mechanism configurable to detect, monitor, and/or manage flora in one or more of a pulmonary tract, a genito-urinary tract or a digestive tract.

21. The apparatus of claim 15, further comprising:
the detect failure to maintain regimen mechanism configurable at least partially as an inorganic device.

22. The apparatus of claim 15, further comprising:
the detect failure to maintain regimen mechanism configurable at least partially as an organic device.

23. The apparatus of claim 15, wherein the failure to maintain regimen mechanism is configured to sense at least one fluid(s) and/or element(s), the at least one fluid(s) and/or element(s) includes at least one from a group, the group including blood, lymph, serum, urine, semen, digestive fluids, tears, amniotic fluid, ambiotic fluid, saliva, mucous, cerebro-spinal fluid, intestinal contents, bile, epithelial exudate, or esophageal contents, synthetic or introduced liquids, such as blood substitutes or drug, nutrient, or buffered saline solutions, liquids containing dissolved gases or gas bubbles, or gases containing fine liquid droplets or solid particles, gases or gaseous mixtures found within the body that may include inhaled and exhaled air, such as in the nasal or respiratory tract, or intestinal gases.

24. A system comprising:
a portion configurable to analyze at least one condition of at least one fluid(s) and/or element(s) as contained within an individual; and
a portion configurable to detect a failure to maintain a regimen for the individual based at least in part on the analyzing the at least one condition of the at least one fluid(s) and/or element(s) as contained within the individual.

25. A method, comprising:
detecting a failure to maintain a regimen for an individual at least partially by transferring at least some analyzing information relating to the detecting the failure to maintain the regimen from within the individual to outside of the individual.

26. The method of claim 25, wherein the detecting a failure to maintain a regimen for an individual at least partially by transferring at least some analyzing information relating to the detecting the failure to maintain the regimen from within the individual to the outside of the individual comprises:
detecting the failure to maintain the regimen for the individual at least partially by transferring at least some element analyzing information relating to the detecting the failure to maintain the regimen for at least one element from within the individual to outside of the individual.

27. The method of claim 25, wherein the detecting a failure to maintain a regimen for an individual at least partially by transferring at least some analyzing information relating to the detecting the failure to maintain the regimen from within the individual to outside of the individual comprises:
detecting the failure to maintain the regimen for the individual at least partially by transferring at least some anesthesia analyzing information relating to the detecting the failure to maintain the regimen for at least one anesthesia from within the individual to the outside of the individual.

28. The method of claim 25, wherein the detecting a failure to maintain a regimen for an individual at least partially by transferring at least some analyzing information relating to the detecting the failure to maintain the regimen from within the individual to outside of the individual comprises:
detecting the failure to maintain the regimen for the individual at least partially by transferring at least some liquid analyzing information relating to the detecting the failure to maintain the regimen for at least one liquid from within the individual to outside of the individual.

29. The method of claim 25, wherein the detecting a failure to maintain a regimen for an individual at least partially by transferring at least some analyzing information relating to the detecting the failure to maintain the regimen from within the individual to outside of the individual comprises:
detecting the failure to maintain the regimen for the individual at least partially by transferring at least some gas analyzing information relating to the detecting the failure to maintain the regimen for at least one gas from within the individual to the outside of the individual.

30. The method of claim 25, further comprising:
treating the individual at least partially outside of the individual at least partially in response to the transferring the at least some analyzing information.

31. The method of claim 25, further comprising treating the individual for the failure to maintain the regimen at least partially in response to the detecting the failure to maintain the regimen for the individual at least partially by transferring at least some liquid or gas analyzing information relating to the detecting the failure to maintain the regimen for at least one liquid or gas from within the individual to the outside of the individual.

32. The method of claim 25, further comprising treating the individual for the failure to maintain the regimen potentially in an absence of a physician or other medical care-providing person.

33. The method of claim 25, further comprising providing a feedback that can maintain the regimen based, at least in part, on a detected condition of the individual, at least one fluid(s) and/or element(s) associated with the individual.

34. The method of claim 25, further comprising providing a medical treatment that is at least partially responsive to a detected condition of the individual, at least one fluid(s) and/or element(s).

35. An apparatus, comprising:
a detecting a failure to maintain regimen mechanism operable to detect a failure to maintain a regimen for an individual at least partially by transferring at least some analyzing information relating to the detecting the failure to maintain the regimen from within the individual to outside of the individual.

36. An apparatus, comprising:
detecting a failure to maintain regimen means for detecting a failure to maintain a regimen for an individual at least partially by transferring at least some analyzing information relating to the detecting the failure to maintain the regimen from within the individual to the outside of the individual.

37. The apparatus of claim 36, further comprising:
feedback means for providing a feedback that can maintain the regimen based, at least in part, on the detecting the failure to maintain regimen means for detecting the failure to maintain the regimen.
